(19) United States
(12) Patent Application Publication
(10) Pub. No.: US 2008/0058351 A1
(43) Pub. Date: Mar. 6, 2008

(54) Novel heterobicyclic compounds

(75) Inventors: Roger Tung, Lexington, MA (US); Scott Harbeson, Cambridge, MA (US)

Correspondence Address: EDWARDS ANGELL PALMER & DODGE LLP, P.O. BOX 55874, BOSTON, MA, 02205, US

(73) Assignee: Concert Pharmaceuticals Inc., Lexington, MA (US)

(21) Appl. No.: 11/824,285

(22) Filed: Jun. 29, 2007

Related U.S. Application Data
(60) Provisional application No. 60/817,759, filed on Jun. 30, 2006.
(60) Provisional application No. 60/904,069, filed on Feb. 27, 2007.

(51) Int. Cl. A61K 31/519; A61P 43/00; C07D 487/04

(52) U.S. Cl. 514/259.3; 544/281

(57) ABSTRACT

This invention relates to novel pyrazolo-pyrimidine compounds and their use as analytical tools and in methods of treating neurological disorders, including sleep disorders such as insomnia.

### RELATED APPLICATIONS

This application claims priority to U.S. Provisional Patent Application No. 60/817,759 filed Jun. 30, 2006 and U.S. Provisional Patent Application No. 60/904,069 filed Feb. 27, 2007.

### BACKGROUND OF THE INVENTION

The term “insomnia” is used to describe all conditions related to the perception of inadequate or non-restful sleep by the patient (Dement, International Pharmacopsychiatry 17:3-38, 1982). Insomnia is the most frequent complaint, being reported by 32% of the adult population surveyed in the Los Angeles area (Bixler et al, Amer J Psychiatry 136:1257-1262, 1979), and 13% of the population surveyed in San Marino, Italy (Lugaresi et al., Psychiatric Annals 17:446-453, 1987). Fully 45% of the surveyed adult population of Alachua County, Fla., reported trouble getting to sleep or staying asleep (Karacan et al., Social Science and Medicine 10:239-244, 1976). The prevalence of insomnia has also been shown to be related to the age and sex of the individuals, being higher in older individuals and in females.

Insomnia, if left untreated, may result in disturbances in metabolism and overall body function. These include reduced productivity and significant changes in mood, behavior and psychomotor function. Chronic insomnia is associated with a higher incidence of morbidity and mortality. Traditionally, the management of insomnia includes treatment and/or mitigation of the etiological factors, improving sleep hygiene and the administration of hypnotic agents. The early hypnotic agents, such as barbiturates, while effective, elicited a spectrum of unwanted side effects and longer-term complications. For example, barbiturates have the potential to result in lethargy, confusion, depression and a variety of other residual effects many hours post dosing, as well as having a potential for being highly addictive.

During the 1980's, the pharmaceutical treatment of insomnia shifted away from barbiturates and other CNS depressants toward the benzodiazepine class of sedative-hypnotics. This class of sedative-hypnotic agents showed substantial effectiveness in producing a calming effect which results in sleep-like states in man and animals (Gee et al., Drugs in Central Nervous Systems, Horwell (ed.), New York, Marcel Dekker, Inc., 1985, p. 123-147) and had a greater safety margin than prior hypnotics, barbiturates or chloral hydrate (Cook and Sepinwall, Mechanism of Action of Benzodiazepines, Costa and Greengard (eds.), New York, Raven Press, 1975, p. 1-28). The therapeutic action of benzodiazepines is believed to be mediated by binding to a specific receptor on benzodiazepine GABA complexes in the brain. As a result of this binding, synaptic transmission is altered at neurons containing the benzodiazepine GABA complex (Clody et al., Benzodiazepines II, Rechtschaffen and Kales (eds.), New York, Springer-Verlag, 1989, p. 341-354). The clinical usefulness of different benzodiazepine hypnotics relates largely to their pharmacokinetic differences with regard to this binding and, in particular, to the half-lives of the parent compound and its active metabolites (Finkle, Benzodiazepines II, Rechtschaffen and Kales (eds.), New York, Springer-Verlag, 1989, p. 619-628).

As with barbiturates, however, many benzodiazepines also possess side effects that limit their usefulness in certain patient populations. These problems include synergy with other CNS depressants (especially alcohol), the development of tolerance upon repeat dosing, rebound insomnia following discontinuation of dosing, hangover effects the next day, and impairment of psychomotor performance and memory (Cook and Sepinwall, supra; Hartman, Benzodiazepines II, Rechtschaffen and Kales (eds.), New York, Springer-Verlag, 1989, p. 187-198; Linnoila and Ellinwood, Benzodiazepines II, Rechtschaffen and Kales (eds.), New York, Springer-Verlag, 1989, p. 601-618). Memory impairment, which can include amnesia for events occurring prior to and after drug administration, is of particular concern in the elderly whose cognitive function may already be impaired by the aging process (Ayd, Benzodiazepines II, Rechtschatfen and Kales (eds.), New York, Springer-Verlag, 1989, p. 593-600; Finkle, supra; Linnoila and Ellinwood, supra).

More recently, a new class of agents has undergone development. These agents are non-benzodiazepine compounds, which bind selectively to a specific receptor subtype of the benzodiazepine receptor. This receptor selectivity is thought to be the mechanism by which these compounds are able to exert a robust hypnotic effect, while also demonstrating an improved safety profile relative to the non-selective, benzodiazepine class of agents. The first of these agents to be approved by the United States Food and Drug Administration (FDA) for marketing in the United States was AmbienCR™ (zolpidem tartrate), which is based on the imidazopyridine backbone (see U.S. Pat. Nos. 4,382,938 and 4,460,592). In addition to Ambien, another compound known as Sonata® (zaleplon), which is a pyrazolopyrimidine-based compound, has received FDA approval (see U.S. Pat. No. 4,626,538). Yet another pyrazolopyrimidine-based compound, indiplon(N-methyl-N-[3-[3-(2-thienylcarbonyl)-pyrazolo[1,5-a]pyrimidin-7-yl]phenyl]acetamide) has completed phase III studies and is awaiting FDA approval (see U.S. Pat. Nos. 6,472,528; 6,485,746 and 6,384,221). Other non-benzodiazepine compounds and/or methods for making or using the same have also been reported (see, e.g., U.S. Pat. Nos. 4,521,422; 4,794,185; 4,808,594; 4,847,256; 4,900,836; 5,714,607; 4,654,347; 5,891,891; 5,538,977; and 6,399,621). Each of these documents is incorporated by reference herein in its entirety.

While significant advances have been made in this field, there is still a need in the art for compounds which are effective as sedative or hypnotic agents generally, particularly in the context of treating insomnia.

### BRIEF DESCRIPTION OF THE DRAWINGS

FIG. 1, panels A through C, depict the comparative metabolism of Compound 1 and indiplon at 0.5 μM (panel A), 0.1 μM (panel B) or 0.2 μM (panel C) each incubated with either 1 mg/mL (panels A and B) or 2 mg/mL (panel C) of human microsomes.

### DETAILED DESCRIPTION OF THE INVENTION

In one aspect, the invention provides a compound of Formula I: 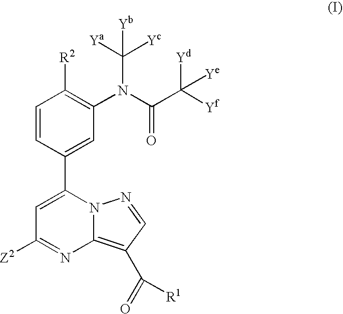
or a pharmaceutically acceptable salt, hydrate, or solvate of said compound, wherein:

each of Y^{a}, Y^{b}, Y^{e}, Y^{d}, Y^{e}, and Y^{f} is independently selected from hydrogen and deuterium, wherein at least one of Y^{a}, Y^{b}, Y^{c}, Y^{d}, and Y^{f} is deuterium;

R¹ is selected from phenyl optionally substituted with 1 to 2 substituents independently selected from halogen, (C₁-C₃)alkoxy, (C₁-C₃)alkyl, (C₁-C₃)alkylamino, (C₁-C₃)dialkylamino, methylenedioxy, (C₁-C₃)alkylsulfonyl, and (C₁-C₃)alkanoylamino; naphthalenyl; furanyl; thiazolyl; biphenyl; thienyl; and pyridinyl, wherein the thiazolyl, biphenyl, thienyl, or pyridinyl is optionally substituted with 1 or 2 substitutents independently selected from halogen, (C₁-C₃)alkoxy and (C₁-C₃)alkyl;

R² is selected from hydrogen, halogen, (C₁-C₃)alkoxy and (C₁-C₃)alkyl; and

Z² is selected from hydrogen and deuterium.

According to one embodiment, at least one of Y^{a}, Y^{b}, Y^{c} is deuterium.

In a more specific embodiment, Y^{a}, Y^{b}, and Y^{c} are simultaneously deuterium.

In another embodiment, at least one of Y^{d}, Y^{e}, and Y^{f} is deuterium.

In another embodiment, Y^{d}, Y^{e}, and Y^{f} are simultaneously deuterium.

In another embodiment, at least two of Y^{a}, Y^{b}, Y^{c}, Y^{d}, Y^{e}, and Y^{f} are deuterium.

In another embodiment, Y^{a}, Y^{b}, Y^{e}, Y^{d}, Y^{e}, and Y^{f} are simultaneously deuterium.

In still another embodiment, R¹ is thienyl. In one aspect, R¹ is thienyl and Y^{a}, Y^{b}, and Y^{c} are simultaneously deuterium. In another aspect, R¹ is thienyl and Y^{d}, Y^{e}, and Y^{f} are simultaneously deuterium. In another aspect, R¹ is thienyl and Y^{a}, Y^{b}, Y^{c}, Y^{d}, Y^{e}, and Y^{f} are simultaneously deuterium.

In one specific embodiment, the invention provides a compound of Formula I having the following structure:

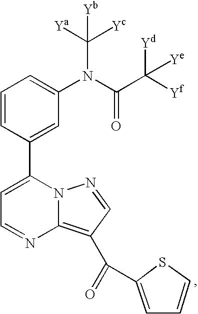

and selected from one of the compounds listed in Table 1 below.

TABLE 1

| Compound | Y^{a} | Y^{b} | Y^{c} | Y^{d} | Y^{e} | Y^{f} |
|---|---|---|---|---|---|---|
| 1 | D | D | D | H | H | H |
| 2 | D | D | D | D | H | H |
| 3 | D | D | D | D | D | H |
| 4 | D | D | D | D | D | D |

or a pharmaceutically acceptable salt, hydrate, or solvate of any of the above compounds.

In other aspects, the invention provides a compound of Formula II: 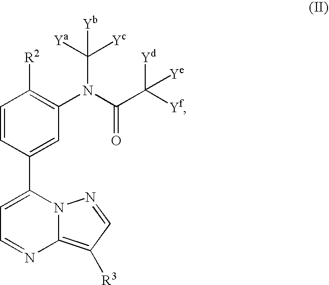
or a pharmaceutically acceptable salt, hydrate, or solvate of said compound, wherein:

each of Y^{a}, Y^{b}, Y^{c}, Y^{d}, Y^{e}, and Y^{f} is independently selected from hydrogen and deuterium, wherein at least one of Y^{a}, Y^{b}, Y^{e}, Y^{d}, Y^{e}, and Y^{f} is deuterium;

R² is selected from hydrogen, halogen, (C₁-C₃)alkoxy and (C₁-C₃)alkyl; and

R³ is selected from hydrogen, cyano, nitro, azido, halogen, and C(O)OR⁴, wherein R⁴ is hydrogen, (C₁-C₃)alkyl, or (C₆-C₂₀)aryl;

In one embodiment, R³ is hydrogen.

In an alternate embodiment, R³ is cyano.

In still another alternated embodiment, R³ is C(O)OR⁴ and R⁴ is (C₁-C₃)alkyl.

In still another aspect, the invention provides a compound of Formula III: 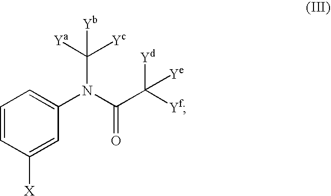
or a pharmaceutically acceptable salt, hydrate, or solvate of said compound, wherein:

each of Y^{a}, Y^{b}, Y^{e}, Y^{d}, Y^{e}, and Y^{f} is independently selected from hydrogen and deuterium, wherein at least one of Y^{a}, Y^{b}, Y^{e}, Y^{d}, Y^{e}, and Y^{f} is deuterium;

X is selected from B(OR⁵)₂, C(O)(CH═CH)ₙN(R⁵)(R⁶), cyano, nitro, azido, and halogen;

each R⁵ is independently selected from hydrogen and (C₁-C₃)alkyl;

R⁶ is selected from hydrogen, (C₁-C₃)alkyl, and (C₆-C₂₀)aryl; and

n is an integer from 0-2.

In one embodiment, the invention provides a compound of formula III wherein X is B(OR⁵)₂.

In a further embodiment, R⁵ is H.

In another embodiment, the invention provides a compound of formula III, wherein X is C(O)(CH═CH)ₙN(R⁵)(R⁶). In a further embodiment, the invention provides a compound of formula III, wherein n is 1.

In other embodiments, the invention provides a compound of formula III, wherein X is C(O)(CH═CH)ₙN(R⁵)(R⁶); R⁵ is (C₁-C₃)alkyl; and R⁶ is (C₁-C₃)alkyl.

Other more specific compounds of the invention, according to formula II or formula III, include the following: 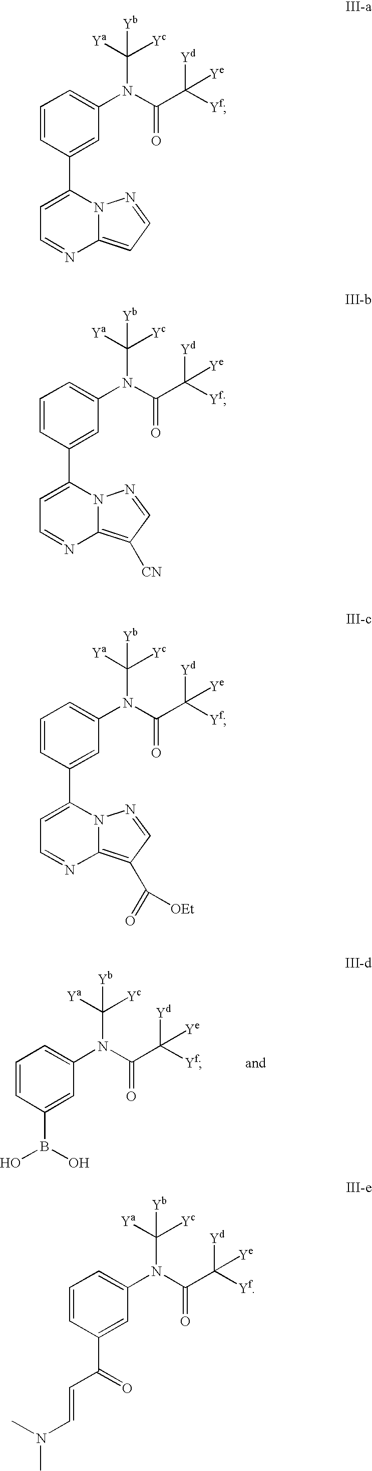
wherein Y^{a}, Y^{b}, and Y^{c} are simultaneously deuterium; and each of Y^{d}, Y^{e}, and Y^{f} are independently selected from hydrogen and deuterium.

In yet another aspect, the invention provides a compound of Formula IV: 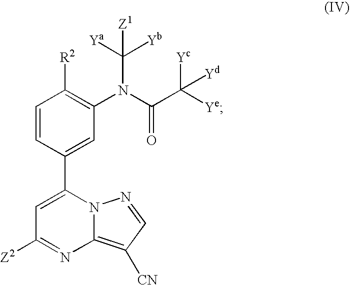
or a pharmaceutically acceptable salt, hydrate, or solvate of said compound, wherein:

each of Y^{a}, Y^{b}, Y^{c}, Y^{d}, and Y^{e} is independently selected from hydrogen and deuterium, wherein at least one of Y^{a}, Y^{b}, Y^{c}, Y^{d}, and Y^{e} is deuterium;

R² is selected from hydrogen, halogen, (C₁-C₃)alkoxy and (C₁-C₃)alkyl;

Z¹ is selected from hydrogen, deuterium, —CH₃; —CH₂D; —CHD₂; and —CD₃; and

Z² is selected from hydrogen and deuterium.

In one embodiment, R² and Z² are simultaneously hydrogen and Z¹ is selected from —CH₃; —CH₂D; —CHD₂; and —CD₃.

In another embodiment, R² and Z² are simultaneously hydrogen and Z¹ is selected form —CD₃ and —CD₃.

In another embodiment, R² and Z² are simultaneously hydrogen, Z¹ is selected from —CH₃ and —CD₃ and Y^{a} and Y^{b} are simultaneously deuterium.

In another embodiment, R² and Z² are each hydrogen, Z¹ is selected from —CH₃ and —CD₃ and Y^{c}, Y^{d}, and Y^{e} are simultaneously deuterium.

In another embodiment, R² and Z² are each hydrogen, Z¹ is selected from —CH₃ and —CD₃ and Y^{c}, Y^{b}, Y^{c}, Y^{d} and Y^{e} are simultaneously deuterium.

Other more specific embodiments of Formula IV are those compounds (Cmpd) wherein R² is H and Y^{a}, Y^{b}, Y^{c}, Y^{d}, Y^{e}, Z¹ and Z² are as defined below in Table 2:

TABLE 2

| Cmpd | Z¹ | Y^{a} | Y^{b} | Y^{c} | Y^{d} | Y^{e} | Z² |
|---|---|---|---|---|---|---|---|
| 100 | —CH₃ | D | D | H | H | H | H |
| 101 | —CH₃ | H | H | D | D | D | H |
| 102 | —CH₃ | H | H | H | H | H | D |
| 103 | —CH₃ | D | D | H | H | H | D |
| 104 | —CH₃ | H | H | D | D | D | D |
| 105 | —CH₃ | D | D | D | D | D | H |
| 106 | —CH₃ | D | D | D | D | D | D |
| 107 | —CD₃ | D | D | H | H | H | H |
| 108 | —CD₃ | H | H | D | D | D | H |
| 109 | —CD₃ | H | H | H | H | H | D |
| 110 | —CD₃ | D | D | H | H | H | D |
| 111 | —CD₃ | H | H | D | D | D | D |
| 112 | —CD₃ | D | D | D | D | D | H |
| 113 | —CD₃ | D | D | D | D | D | D |

In certain embodiments, the invention provides a compound of any one of Formulae I-IV, comprising three or more deuterium atoms. In other embodiments, the invention provides a compound of any one of Formulae I-IV, comprising four or more deuterium atoms.

In another embodiment, any atom not designated as deuterium in any of the embodiments set forth above is present at its naturally abundant isotopic state and each carbon atom is present at its naturally abundant isotopic state.

### Definitions

As used herein, the term “compound of formula I” (or of any of Formulae II-IV), includes the pharmaceutically acceptable salts of said compound, its hydrates, and solvates.

In addition, some of the compounds of this invention have one or more double bonds, or one or more asymmetric centers. Such compounds can occur as racemates, racemic mixtures, single enantiomers, individual diastereomers, diastereomeric mixtures, and cis- or trans- or E- or Z-double isomeric forms. All such isomeric forms of these compounds are expressly included in the present invention. The compounds of this invention may also be represented in multiple tautomeric forms, in such instances, the invention expressly includes all tautomeric forms of the compounds described herein (e.g., alkylation of a ring system may result in alkylation at multiple sites, the invention expressly includes all such reaction products). All such isomeric forms of such compounds are expressly included in the present invention. A compound of the present invention will include not only a stereoisomeric mixture, but also individual respective stereoisomers substantially free from one another stereoisomers. The term “substantially free” as used herein means less than 25% of other stereoisomers, preferably less than 10% of other stereoisomers, more preferably less than 5% of other stereoisomers and most preferably less than 2% of other stereoisomers, are present. Methods of obtaining or synthesizing diastereomers are well known in the art and may be applied as practicable to final compounds or to starting material or intermediates. Other embodiments are those wherein the compound is an isolated compound.

The term “isotopologue” refers to species that differ from a specific compound of this invention only in the isotopic composition of their molecules or ions. The terms “lighter isotopologue” and “lighter atom isotopologue” as used herein, refer to species that differs from a specific compound of this invention in that it comprises one or more light isotopic atoms ¹H or ¹²C at positions designated as a deuterium or ¹³C in that specific compound. For the purposes of this invention, ¹¹C is not referred to as a light isotope of carbon.

A specific compound of this invention may also be referred to as a “heavy atom isotopic compound” to distinguish it from its lighter isotopologues when discussing mixtures of isotopologues.

Chemical naming terminology can be complex and different chemical names can often reasonably be applied to the same structure. To avoid any confusion, compounds of the invention refer to the chemical structure shown herein for such compounds.

The compounds and compositions of this invention are also useful as analytical reagents for determining the concentration of the non-deuterated compounds in solution. “Non-deuterated compounds” as used herein refers to a compound wherein all hydrogen and all carbon atoms are present at their natural isotopic abundance percentages.

It is recognized that some variation of natural isotopic abundance occurs depending upon the origin of chemical materials. Thus, a preparation of indiplon and synthetic intermediates thereof inherently comprise small amounts of deuterated and/or ¹³C-containing isotopologues. The concentration of naturally abundant stable hydrogen and carbon isotopes, notwithstanding this variation, is small and immaterial with respect to the degree of stable isotopic substitution of compounds of this invention. See for instance Wada E and Hanba Y, Seikagaku 1994 66: 15; Ganes L Z et. al., Comp. Biochem. Physiol. A Mol. Integr. Physiol. 1998 119: 725.

In one embodiment, a compound as defined herein, contains less than 10%, preferably less than 6%, and more preferably less than 3% of all other isotopologues. In other embodiments, the compound contains less than X% of all other isotopologues, where X is a number between about 0 and 10, inclusive. A compound of this invention preferably comprises hydrogen and carbon atoms, not specifically designated as deuterium and ¹³C, respectively, in their natural isotopic abundance. In certain embodiments, compositions of matter that contain greater than 10% of all other specific isotopologues combined are referred to herein as mixtures and must meet the parameters set forth below.

In other embodiments, when a particular position is designated as having deuterium in a compound of this invention, it is understood that the abundance of deuterium at that position is substantially greater than the natural abundance of deuterium, which is 0.015%. A position designated as having deuterium typically has a minimum isotopic enrichment factor of at least 3000 (45% deuterium incorporation) at each atom designated as deuterium in said compound.

The term “isotopic enrichment factor” as used herein means the ratio between the isotopic abundance and the natural abundance of a specified isotope.

In still other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition.

A salt of a compound of this invention is formed between an acid and a basic group of the compound, such as an amino functional group, or a base and an acidic group of the compound, such as a carboxyl functional group. According to another preferred embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound or a prodrug of a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric, hydrobromic, hydroiodic, sulfuric and phosphoric acid, as well as organic acids such as para-toluenesulfonic, salicylic, tartaric, bitartaric, ascorbic, maleic, besylic, fumaric, gluconic, glucuronic, formic, glutamic, methanesulfonic, ethanesulfonic, benzenesulfonic, lactic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and the like salts. Preferred pharmaceutically acceptable acid addition salts include those formed with mineral acids such as hydrochloric acid and hydrobromic acid, and especially those formed with organic acids such as maleic acid.

As used herein, the term “alkyl” refers to a straight-chained or branched hydrocarbon group containing the specified number of carbon atoms.

The term “alkoxy” refers to an —O-alkyl radical.

As used herein, the term “halogen” or “halo” means —F, —Cl, —Br or —I.

The term “aryl” refers to a hydrocarbon monocyclic, bicyclic or tricyclic aromatic ring system. Examples of aryl groups include phenyl, naphthyl, anthracenyl, fluorenyl, indenyl, azulenyl, and the like.

The term “alkylamino” refers to an amino substituent which is further substituted with one or two alkyl groups. The term “aminoalkyl” refers to an alkyl substituent which is further substituted with one or more amino groups.

As used herein the term “substituent” or “substituted” means that a hydrogen radical on a compound or group is replaced with another specified group.

As used herein, the term “hydrate” means a compound which further includes a stoichiometric or non-stoichiometric amount of water bound by non-covalent intermolecular forces.

As used herein, the term “solvate” means a compound which further includes a stoichiometric or non-stoichiometric amount of solvent such as water, acetone, ethanol, methanol, dichloromethane, 2-propanol, or the like, bound by non-covalent intermolecular forces.

The term “heavy atom” refers to isotopes of higher atomic weight than the predominant naturally occurring isotope. The term “stable heavy atom” refers to non-radioactive heavy atoms. Both “²H” and “D” refer to deuterium.

“Stereoisomer” refers to both enantiomers and diastereomers. “Nos.” refers to numbers. “AIBN” refers to 2,2′-azo-bis(isobutyronitrile). “Boc” refers to tert-butoxycarbonyl. “PMP” refers to 4-methoxyphenyl. “DHP” refers to dihydropyran. “THP” refers to tetrahydropyran. “THF” refers to tetrahydrofuran. “DMF” refers to N,N-dimethylformamide. “DMSO” refers to dimethylsulfoxide. “aq.” Refers to aqueous. “h” refers to hours. “min” refers to minutes. “tert” refers to tertiary. “brine” refers to saturated aqueous sodium chloride. “US” or “U.S.” refers to the United States of America. “FDA” refers to Food and Drug Administration. “NDA” refers to New Drug Application. “AUC” refers to area under the plasma-time concentration curve.

The invention further provides compositions comprising a mixture of a compound of this invention and its lighter isotopologues. These mixtures may occur, for instance, simply as the result of an inefficiency of incorporating an isotope at a given position; intentional or inadvertent exchange of protons for deuterium, e.g. exchange of bulk solvent for heteroatom-attached deuterium; or intentional mixtures of pure compounds.

In one embodiment, such mixtures comprise at least about 50% of the heavy atom isotopic compound (i.e., less than about 50% of lighter isotopologues). More preferable is a mixture comprising at least 80% of the heavy atom isotopic compound. Most preferable is a mixture comprising 90% of the heavy atom isotopic compound.

In an alternate embodiment the mixture comprises a compound of any of Formulae I-IV and its lighter isotopologues in relative proportions such that at least about 50%, preferably at least 80%, more preferably at least 90%, even more preferably at least 95% and most preferably at least 98% of the compounds in said mixture comprise a heavy atom isotope at each position containing a stable heavy atom isotope in the heavy atom isotopic compound.

### Pharmaceutical Compositions

The invention also provides compositions comprising an effective amount of a compound of the invention any of formulae I, II, and IV; and an acceptable carrier. The carrier(s) must be “acceptable” in the sense of being compatible with the other ingredients of the formulation.

In a preferred embodiment, the invention provides a composition comprising a compound of any of formulae I, II, and IV; and a pharmaceutically acceptable carrier, wherein said composition is formulated for pharmaceutical use (“a pharmaceutical composition”). A “pharmaceutically acceptable carrier” is a carrier that is compatible with the other ingredients of the composition and not deleterious to the recipient thereof in amounts typically used in medicaments.

In one aspect, the pharmaceutical composition is for use in the treatment of a neurological disorder, sleep disorder, a sleep/wake disorder, anxiety, depression, or attention deficit disorder.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets and sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers or both, and then if necessary shaping the product.

In certain preferred embodiments, the compound is administered orally. Compositions of the present invention suitable for oral administration may be presented as discrete units such as capsules, sachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added. Surfactants such as sodium lauryl sulfate may be useful to enhance dissolution and absorption.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as Ph. Helv or a similar alcohol.

The pharmaceutical compositions of this invention may be administered in the form of suppositories for rectal or vaginal administration. These compositions can be prepared by mixing a compound of Formula I with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is especially useful when the desired treatment involves areas or organs readily accessible by topical application. For application topically to the skin, the pharmaceutical composition will be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches and iontophoretic administration are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g., Rabinowitz J D and Zaffaroni A C, U.S. Pat. No. 6,803,031, assigned to Alexza Molecular Delivery Corporation.

Application of the subject therapeutics may be local, so as to be administered at the site of interest. Various techniques can be used for providing the subject pharmaceutical compositions at the site of interest, such as injection, use of catheters, trocars, projectiles, pluronic gel, stents, sustained drug release polymers or other device which provides for internal access.

Thus, according to another embodiment, a compound of any of formulae I, II, and IV may be incorporated into a pharmaceutical composition for coating an implantable medical device, such as prostheses, artificial valves, vascular grafts, stents, or catheters. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings are optionally further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

Also within the scope of this invention are pharmaceutical compositions comprising an effective amount of a compound of any of formulae I, II, and IV in combination with an effective amount of a second therapeutic agent and a pharmaceutically acceptable carrier. The second therapeutic agent may be one that is useful for treating a sleep disorder such as insomnia; or an agent that is useful to treat a disease or condition in which insomnia or another sleep disorder is a known symptom. One example of such a disease or condition is depression. Such second therapeutic agents that may be formulated in combination with the compounds of this invention include, but are not limited to, serotonin reuptake inhibitors (SSRIs), dopamine reuptake inhibitors (SNRIs), and other non-benzodiazepine hypnotics.

In another embodiment, the invention provides separate dosage forms of a compound of any of formulae I, II, and IV and a second therapeutic agent, wherein said compound and said second therapeutic agent are associated with one another. The term “associated with one another” as used herein means that the separate dosage forms are packaged together in the same container (e.g., in separate blister packs attached to one another, in separate compartments of a compartmentalized container, in separate vessels contained in the same box, etc.), or otherwise attached to one another such that it is readily apparent that the separate dosage forms are intended to be sold and administered together (within less than 24 hours of one another, consecutively or simultaneously).

In the pharmaceutical compositions of the invention, a compound of any of formulae I, II, and IV is present in an effective amount. As used herein, the term “effective amount” refers to an amount which, when administered in a proper dosing regimen, is sufficient to reduce or ameliorate the severity, duration, or enhance function compromised by a sleep disorder, including insomnia.

The interrelationship of dosages for animals and humans (based on milligrams per meter squared of body surface) is described in Freireich et al., (1966) Cancer Chemother Rep 50: 219. Body surface area may be approximately determined from height and weight of the patient. See, e.g., Scientific Tables, Geigy Pharmaceuticals, Ardley, N.Y., 1970, 537. An effective amount of a compound of Formula I, II, or IV can range from about 0.001 mg/kg to about 500 mg/kg, more preferably 0.01 mg/kg to about 50 mg/kg, yet more preferably 0.025 mg/kg to about 1.5 mg/kg. Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician.

In other embodiments, the invention provides a pharmaceutical composition in dosage unit form comprising from 0.1 to 250 mg of a compound of formula I, II, or IV. In a further embodiment, the invention provides a pharmaceutical composition in dosage unit form comprising from 2 to 50 mg of a compound of formula I, II, or IV.

It is expected that some of the second therapeutic agents will act synergistically with the compounds of this invention. When this occurs, it will allow the effective dosage of the second therapeutic agent and/or the compound of any of formulae I, II, and IV to be reduced from that required in a monotherapy. This has the advantage of minimizing toxic side effects of either the second therapeutic agent or a compound of Formula I, synergistic improvements in efficacy, improved ease of administration or use and/or reduced overall expense of compound preparation or formulation.

In each of the above embodiments, the second therapeutic agent or agents may be administered together with a compound of any of formulae I, II, and IV as part of a single dosage form or as separate dosage forms. Alternatively, the second therapeutic agent or agents may be administered prior to, consecutively with, or following the administration of a compound of any of formulae I, II, and IV. In such combination therapy treatment, both the compounds of this invention and the second therapeutic agent(s) are administered by conventional methods. The administration of the second therapeutic agent(s) may occur before, concurrently with, and/or after the administration of the compound of any of formulae I, II, and IV. When the administration of the second therapeutic agent occurs concurrently with a compound of any of formulae I, II, and IV, the two (or more) agents may be administered in a single dosage form (such as a composition of this invention comprising a compound of any of formulae I, II, and IV, a second therapeutic agent or agents as described above, and a pharmaceutically acceptable carrier), or in separate dosage forms. The administration of a composition of this invention comprising both a compound of any of formulae I, II, and IV and a second therapeutic agent(s) to a subject does not preclude the separate administration of said second therapeutic agent(s), any other therapeutic agent or any compound of this invention to said subject at another time during a course of treatment.

Effective amounts of second therapeutic agent or agents useful in the methods of this invention are well known to those skilled in the art and guidance for dosing may be found in patents referenced herein, as well as in Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the optimal effective-amount range of the additional agent(s).

### Synthesis of Compounds of the Invention

Compounds of the invention can be made by means known in the art of organic synthesis. For instance, routes to the all-hydrogen isotopologues of compounds of this invention are described in U.S. Pat. No. 6,399,621; U.S. Pat. No. 4,521,422; U.S. Pat. No. 4,900,836; U.S. Pat. No. 6,472,528; and Sorbera, L. A. et al., Drugs Fut, 2003, 28:739; which are incorporated herein in their entirety.

Methods of incorporating deuterium in target compounds are extensively documented. See, for instance, The Journal of Labelled Compounds and Radiopharmaceuticals (John Wiley & Sons), most issues of which contain detailed experimental descriptions on specific incorporation of deuterium into bioactive small organic molecules. See also, for instance, Leis H J, Curr Org Chem, 1998, 2:131 and reference therein, and Moebius G, ZfI-Mitteilungen 1989, 150:297. Suitable commercial supplies of deuterium-labeled reagents include, among others, Isotec, Inc. (Miamisburg, Ohio); Cambridge Isotope Laboratories (Andover, Mass.); ICON Services Inc. (Summit, N.J.); and C/D/N Isotopes, Inc. (Pointe-Claire, Quebec, Canada).

Methods for optimizing reaction conditions, if necessary minimizing competing by-products, are known in the art. Reaction optimization and scale-up may advantageously utilize high-speed parallel synthesis equipment and computer-controlled microreactors (e.g. Design And Optimization in Organic Synthesis, 2^{nd} Edition, Carlson R, Ed, 2005; Elsevier Science Ltd.; Jähnisch, K et al, Angew. Chem. Int. Ed. Engl. 2004 43: 406; and references therein). Additional reaction schemes and protocols may be determined by the skilled artesian by use of commercially available structure-searchable database software, for instance, SciFinder® (CAS division of the American Chemical Society) and CrossFire Beilstein® (Elsevier MDL), or by appropriate keyword searching using an internet search engine such as Google® or keyword databases such as the US Patent and Trademark Office text database.

In each of the synthesis schemes depicted below an asterisk (*) is used to denote optional deuteration of the indicated compound.

In an exemplary synthetic method, condensation of 2-acetylthiophene (A) with dimethylformamide dimethylacetal produces enaminone B, which is then cyclized to the isoxazole C by treatment with hydroxylamine. Further condensation of C with dimethylformamide dimethylacetal, with concomitant isoxazole ring opening, gives the enamino nitrile D, which is then cyclized with aminoguanidine nitrate E under basic conditions to furnish the aminopyrazole F. 3-(acetamido)acetophenone G is condensed with dimethylformamide dimethylacetal to give enaminone H, which is then alkylated at the amide nitrogen by means of optionally deuterated iodomethane and NaH to give compound *J. Finally, compound *J is condensed with the aminopyrazole F in refluxing AcOH to provide deuterated compounds *K of the invention (Schemes 1 and 2).

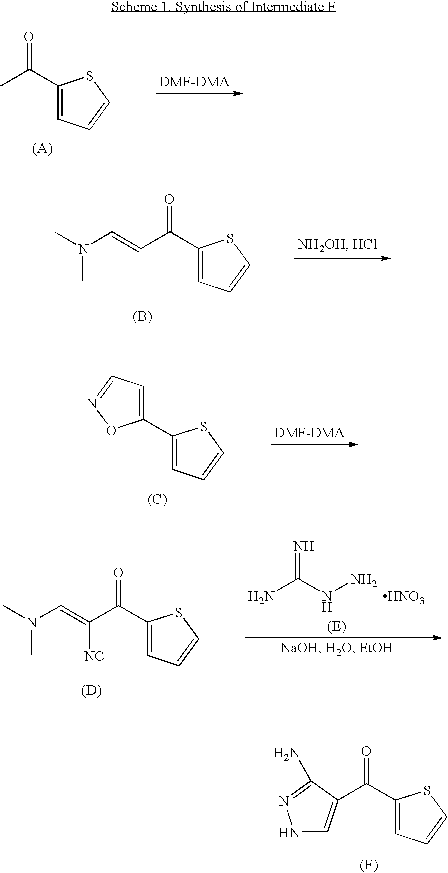

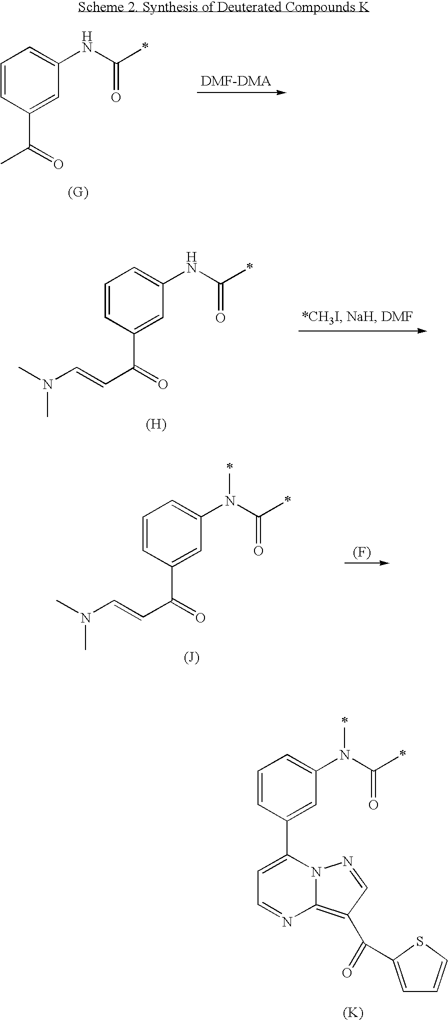

An alternate approach to making deuterated compounds (K) is shown in Scheme 3 below. Bromination of 5-aminopyrazole (L) with Br₂ in AcOH gives 4-bromo-5-aminopyrazole (M), which is cyclized with *J (from Scheme 2) in AcOH to yield the 3-bromopyrazolo[1,5-a]pyrimidine derivative *N. Finally, this compound can be condensed with 2-thienylboronic acid (P) and CO by means of a Pd(0) catalyst to provide *K.

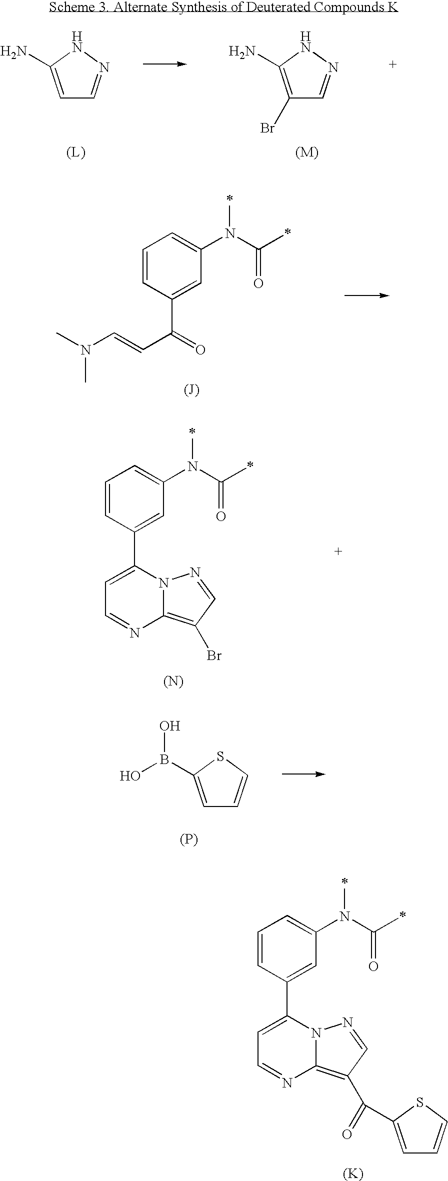

Another approach to making deuterated compounds K is shown in Scheme 4 below. Cyclization of L with the acetamide *J in AcOH gives the pyrazolo[1,5-a]pyrimidine derivative *Q, which is finally acylated with 2-thienylcarbonyl chloride (R) by means of AlCl₃ to provide product *K.

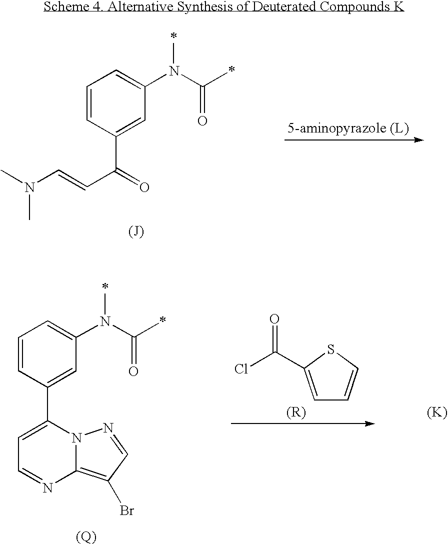

Another approach to making deuterated compounds K is shown in Scheme 5 below. Cyclization of ethoxymethylenemalonodinitrile (S) with hydrazine gives 5-aminopyrazole-4-carbonitrile (T), which is further cyclized with acetamide (*J) in refluxing AcOH to yield the 3-cyanopyrazolo[1,5-a]pyrimidine derivative U. Finally, U is condensed with 2-bromothiophene (V) by means of Mg to provide compound K.

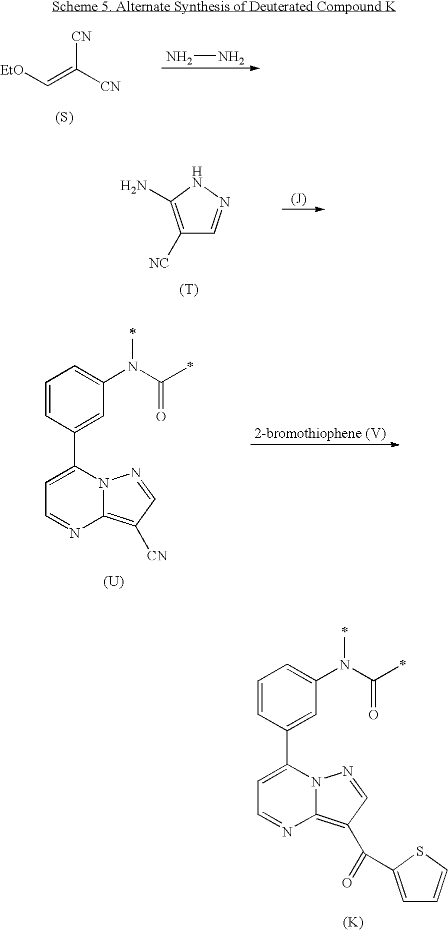

Another approach to making deuterated compounds K is shown in Scheme 6 below. Cyclization of 5-aminopyrazole-4-carboxylic acid ethyl ester (W) with either, 3,3-diethoxypropionic acid ethyl ester or 3-oxopropionic acid ethyl ester in refluxing AcOH gives 7-hydroxypyrazolo[1,5-a]pyrimidine-3-carboxylic acid ethyl ester Y, which is treated with POCl₃ to yield the corresponding chloro derivative Z. Condensation of compound Z with *3-(N-methylacetamido)phenylboronic acid (*AA) by means of Pd(PPh₃)₄ and Na₂CO₃ in ethanol affords the pyrazolo[1,5-a]pyrimidine-carboxylic acid ethyl ester BB, which is finally condensed with V and Mg to provide compound K.

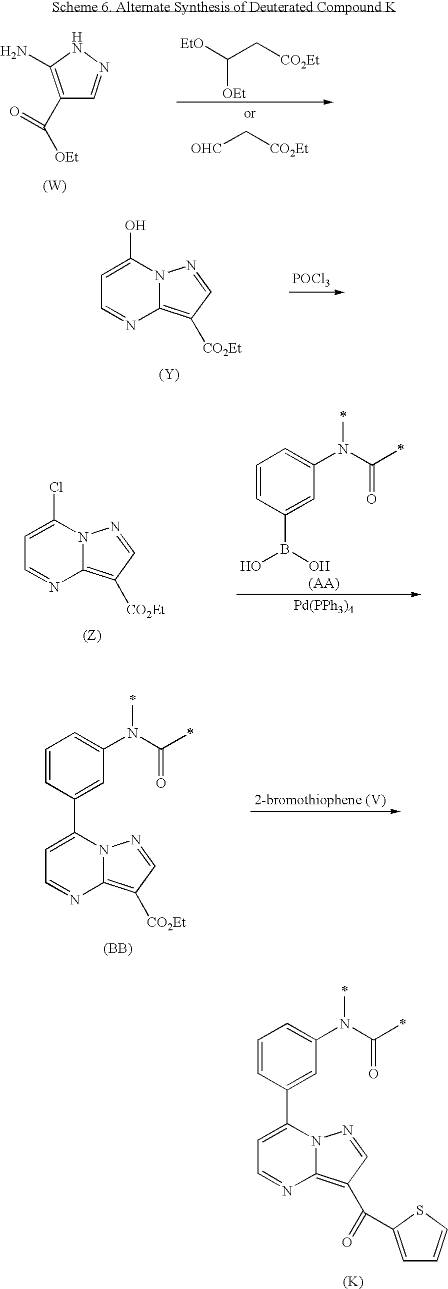

Another approach to making deuterated compounds K is shown in Scheme 7 below. Commercially available compound XII is condensed with the aminopyrazole XIII to yield the 5-oxo-4,5-dihydropyrazolo[1,5-a]pyrimidine XIV according to the method of Reimlinger H et al., Chem. Ber. 1970, 103, p. 3252. This compound is then halogenated according to European Patent Office patent publication no. EP 1666468, using phosphorylchloride and N,N-diethylaniline at elevated temperature to yield chloro-derivative XV. The halogen can be reductively cleaved with the concomitant reduction of the nitro group to the amine, e.g., with deuterium gas and palladium/carbon catalyst to yield the 7-deutero analog XVI as described by Buchini S et al., Eur. J. Org. Chem. 2006, 14, p. 3152. The aniline can then be reacted with acetic anhydride or deuterated acetic anhydride under basic conditions to provide XVI. Treatment of the amide with sodium hydride in DMF followed by ethyl iodide or the appropriate commercially available deuterated ethyl iodide provides compounds of formula I. See, e.g., Dusza, J P et al., U.S. Pat. No. 4,626,538.

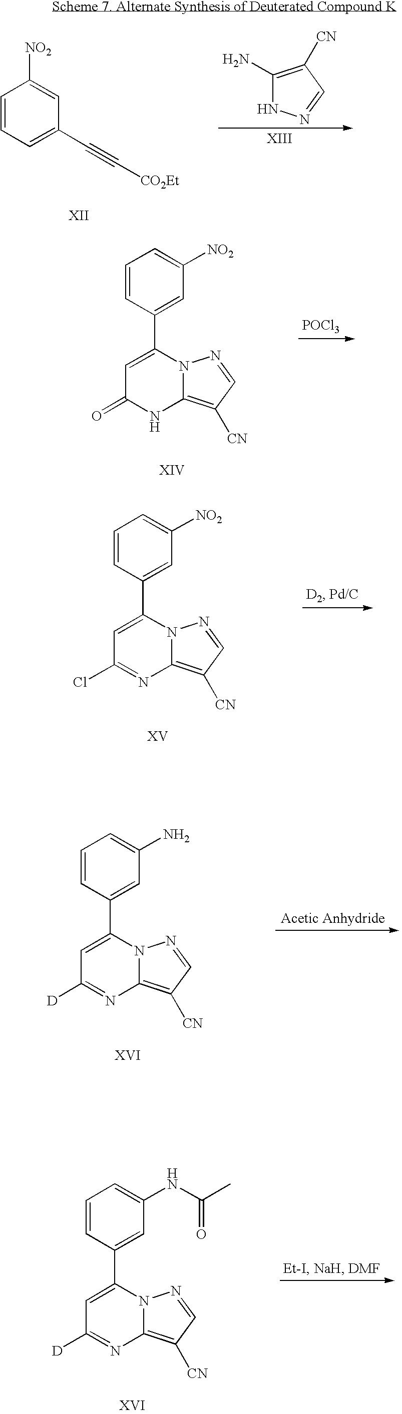

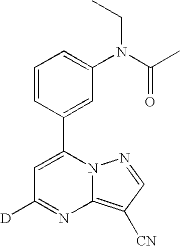

The synthetic methods described herein may also additionally include steps, either before or after any of the steps described in Schemes 1 to 7, to add or remove suitable protecting groups in order to ultimately allow synthesis of the compound of the formulae described herein. The methods delineated herein contemplate converting compounds of one formula to compounds of another formula. The process of converting refers to one or more chemical transformations, which can be performed in situ, or with isolation of intermediate compounds. The transformations can include reacting the starting compounds or intermediates with additional reagents using techniques and protocols known in the art, including those in the references cited herein. Certain intermediates can be used with or without purification (e.g., filtration, distillation, sublimation, crystallization, trituration, solid phase extraction, chromatography, etc.).

Combinations of substituents and variables envisioned by this invention are only those that result in the formation of stable compounds. The term “stable”, as used herein, refers to compounds which possess stability sufficient to allow manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition related to sleep disorders).

### Methods of Treatment

The present invention provides a method for treating conditions which benefit from administration of agents which possess anxiolytic, anti-anoxic, sleep-inducing, hypnotic, anticonvulsant, and/or skeletal muscle relaxant properties. Such conditions include insomnia specifically, as well as sleep disorders generally and other neurological and psychiatric complaints, anxiety states, vigilance disorders, such as for combating behavioral disorders attributable to cerebral vascular damage and to the cerebral sclerosis encountered in geriatrics, epileptic vertigo attributable to cranial trauma, and for metabolic encephalopathies.

Methods are provided for the treatment of anxiety, depression, a sleep disorder, attention deficit disorder, or Alzheimer's dementia, comprising administering to a patient in need of such treatment a therapeutically effective amount of a compound as described above. The patient may be a human or other mammal. Treatment of humans, domesticated companion animals (pets) or livestock animals suffering from certain CNS disorders with an effective amount of a compound of the invention is encompassed by the present invention.

The present invention also provides methods for potentiating a therapeutic effect of a CNS agent, comprising administering to a patient a CNS agent and a compound as described above.

Within certain aspects, the present invention provides methods for inhibiting the development of a CNS disorder. In other words, therapeutic methods provided herein may be used to treat a disorder, or may be used to prevent or delay the onset of such a disease in a patient who is free of detectable CNS disorder. CNS disorders are discussed in more detail below, and may be diagnosed and monitored using criteria that have been established in the art. Patients may include humans, domesticated companion animals (pets, such as dogs) and livestock animals, with dosages and treatment regimes as described above.

CNS disorders that can be treated using compounds and compositions provided herein include: Depression, e.g., depression, a typical depression, bipolar disorder, depressed phase of bipolar disorder. Anxiety, e.g., general anxiety disorder (GAD), agoraphobia, panic disorder±agoraphobia, social phobia, specific phobia, Post traumatic stress disorder, obsessive compulsive disorder (OCD), dysthymia, adjustment disorders with disturbance of mood and anxiety, separation anxiety disorder, anticipatory anxiety acute stress disorder, adjustment disorders, cyclothymia. Sleep disorders, e.g., sleep disorders including primary insomnia, circadian rhythm sleep disorder, dyssomnia NOS, parasomnias, including nightmare disorder, sleep terror disorder, sleep disorders secondary to depression and/or anxiety or other mental disorders, substance induced sleep disorder. Cognition Impairment, e.g., cognition impairment, Alzheimer's disease, Parkinson's disease, mild cognitive impairment (MCI), age-related cognitive decline (ARCD), stroke, traumatic brain injury, AIDS associated dementia, and dementia associated with depression, anxiety or psychosis. Attention Deficit Disorder, e.g., attention deficit disorder (ADD), and attention deficit and hyperactivity disorder (ADHD).

In a specific embodiment of this invention, an effective amount of a compound of the invention is administered to a patient as a sedative or hypnotic agent, particular in the context of treatment of insomnia. Compounds of the invention have been found to have particular advantageous properties in this context.

In a preferred embodiment, the present invention provides a method for the prevention or treatment of a circadian rhythm disorder in a mammal, including time-zone change (jet-lag) syndrome, shift-work sleep disorder, delayed sleep-phase syndrome, advanced sleep-phase syndrome, and non-24-hour sleep-wake disorder, which comprises administering to the mammal an effective amount of a compound of the invention.

In another embodiment, the present invention provides a method for shortening the time of reentrainment of circadian rhythms in a subject following a shift in the sleep-wake cycle which comprises administering to the subject an appropriate amount of a compound of the present invention.

In another embodiment, the present invention provides a method for alleviating the effects of jet lag in a traveler, especially a mammal, which comprises administering to the traveler an alertness increasing amount of a compound of the instant invention. The purpose of this embodiment is to assist the body to adjust physiologically to the changes in sleep and feeding patterns when crossing several time zones.

In another embodiment, the present invention provides a method for resetting the internal circadian clock in a subject, for example shift workers changing from a day to a night shift or vice versa, which comprises administering to the subject an appropriate amount of a compound of the instant invention.

The present invention is further directed to the use of compounds of the invention for enhancing or improving sleep quality as well as preventing and treating sleep disorders and sleep disturbances in a mammal. In particular, the present invention provides a method for enhancing or improving sleep quality by increasing sleep efficiency and augmenting sleep maintenance. In addition, the present invention provides a method for preventing and treating sleep disorders and sleep disturbances in a mammal which comprising the administration of a compound of the invention.

The following outcomes in a subject which are provided by the present invention may be correlated to enhancement in sleep quality: an increase in the value which is calculated from the time that a subject sleeps divided by the time that a subject is attempting to sleep; a decrease in sleep latency (the time it takes to fall asleep); a decrease in the number of awakenings during sleep; a decrease in the time spent awake following the initial onset of sleep; an increase in the total amount of sleep; an increase the amount and percentage of REM sleep; an increase in the duration and occurrence of REM sleep; a reduction in the fragmentation of REM sleep; an increase in the amount and percentage of slow-wave (i.e. stage 3 or 4) sleep; an increase in the amount and percentage of stage 2 sleep; a decrease in the number of awakenings, especially in the early morning; an increase in daytime alertness; and increased sleep maintenance. Secondary outcomes which may be provided by the present invention include enhanced cognitive function and increased memory retention.

The present invention is further useful for the prevention and treatment of sleep disorders and sleep disturbances including sleep problems associated with insomnia, hypersomnia, sleep apnea, narcolepsy, nocturnal myoclonus, REM sleep interruptions, jet-lag, shift workers' sleep disturbances, dyssomnias, night terror, insomnias associated with depression or with emotional/mood disorders, dysfunctions associated with sleep (parasomnias), as well as sleep walking and enuresis, as well as sleep disorders which accompany aging. Sleep disorders and sleep disturbances are generally characterized by difficulty in initiating or maintaining sleep or in obtaining restful or enough sleep.

In addition, certain drugs may also cause reductions in REM sleep as a side effect and the present invention may be used to correct those types of sleeping disorders as well. The present invention would also be of benefit in the treatment of syndromes such as fibromyalgia which are manifested by non-restorative sleep and muscle pain or sleep apnea which is associated with respiratory disturbances during sleep. It will be clear to one skilled in the art that the present invention is not limited to just sleep disorders and sleep disturbances, but is applicable to a wide variety of conditions which result from a diminished quality of sleep.

In the present invention, it is preferred that the subject mammal is a human. Although the present invention is applicable both old and young people, it may find greater application in elderly people. Further, although the invention may be employed to enhance the sleep of healthy people, it may be especially beneficial for enhancing the sleep quality of people suffering from sleep disorders or sleep disturbances.

There are a number of ways to determine whether the onset, duration or quality of sleep (e.g. non-restorative or restorative sleep) is impaired or improved. One method is a subjective determination of the patient, e.g., do they feel drowsy or rested upon waking. Other methods involve the observation of the patient by another during sleep, e.g., how long it takes the patient to fall asleep, how many times does the patient wake up during the night, how restless is the patient during sleep, etc. Another method is to objectively measure the stages of sleep.

Polysomnography is the monitoring of multiple electrophysiological parameters during sleep and generally includes measurement of EEG activity, electrooculographic activity and electromyographic activity, as well as other measurements. These results, along with observations, can measure not only sleep latency (the amount of time required to fall asleep), but also sleep continuity (overall balance of sleep and wakefulness) which may be an indication of the quality of sleep.

There are five distinct sleep stages which can be measured by polysomnography: rapid eye movement (REM) sleep and four stages of no-rapid eye movement (NREM) sleep (stages 1, 2, 3 and 4). Stage 1 NREM sleep is a transition from wakefulness to sleep and occupies about 5% of time spent asleep in healthy adults. Stage 2 NREM sleep, which is characterized by specific EEG waveforms (sleep spindles and K complexes), occupies about 50% of time spent asleep. Stages 3 and 4 NREM sleep (also known collectively as slow-wave sleep) are the deepest levels of sleep and occupy about 10-20% of sleep time. REM sleep, during which the majority of typical story like dreams occur, occupies about 20-25% of total sleep.

These sleep stages have a characteristic temporal organization across the night. NREM stages 3 and 4 tend to occur in the first one-third to one-half of the night and increase in duration in response to sleep deprivation. REM sleep occurs cyclically through the night alternating with NREM sleep about every 80-100 minutes. REM sleep periods increase in duration toward the morning. Human sleep also varies characteristically across the life span. After relative stability with large amounts of slow-wave sleep in childhood and early adolescence, sleep continuity and depth deteriorate across the adult age range. This deterioration is reflected by increased wakefulness and stage 1 sleep and decreased stages 3 and 4 sleep.

In a separate aspect, the present invention provides methods for potentiating the action (or therapeutic effect) of other CNS agent(s). Such methods comprise administering an effective amount of a compound provided herein in combination with another CNS agent. Such CNS agents include, but are not limited to the following: for anxiety, serotonin receptor agonists and antagonists; for anxiety and depression, neurokinin receptor antagonists or corticotropin releasing factor receptor (CRF₁) antagonists; for sleep disorders, melatonin receptor agonists; and for neurodegenerative disorders, such as Alzheimer's dementia, nicotinic agonists, muscarinic agents, acetylcholinesterase inhibitors and dopamine receptor agonists. Within preferred embodiments, the present invention provides a method of potentiating the antidepressant activity of selective serotonin reuptake inhibitors (SSRIs) by administering an effective amount of a compound of any one of Formulae I, II or IV in combination with an SSRI. An effective amount of compound is an amount sufficient to result in a detectable change in patient symptoms, when compared to a patient treated with the other CNS agent alone.

Methods for determining the presence or absence of GABA_{A} receptor in a sample are further provided, comprising: (a) contacting a sample with a compound as described above under conditions that permit binding of the compound to GABA_{A} receptor; and (b) detecting a level of compound bound to GABA_{A} receptor. Furthermore, the compounds as described above can be radiolabeled, wherein the step of detection comprises: (i) separating unbound compound from bound compound; and (ii) detecting the presence or absence of bound compound in the sample.

The present invention further provides a method for altering the signal-transducing activity of GABA_{A} receptor, comprising contacting a cell expressing GABA_{A} receptor with a compound as described above in an amount sufficient to detectably alter the electrophysiology of the cell.

Additionally, the cell recombinantly expresses a heterologous GABA_{A} receptor, wherein the alteration of the electrophysiology of the cell is detected by intracellular recording or patch clamp recording.

The compounds provided herein detectably alter (modulate) ligand binding to GABA_{A} receptor, as determined using a standard in vitro receptor binding assay. References herein to a “GABA_{A} receptor ligand binding assay” are intended to refer to the standard in vitro receptor binding assay. Briefly, a competition assay may be performed in which a GABA_{A} receptor preparation is incubated with labeled (e.g., ³H) ligand, such as Flumazenil, and unlabeled test compound. Incubation with a compound that detectably modulates ligand binding to GABA_{A} receptor will result in a decrease or increase in the amount of label bound to the GABA_{A} receptor preparation, relative to the amount of label bound in the absence of the compound. Preferably, such a compound will exhibit a Ki at a GABA_{A} receptor of less than 1 micromolar, more preferably less than 500 nM, 100 nM, 20 nM or 10 nM. The GABA_{A} receptor used to determine in vitro binding may be obtained from a variety of sources, for example from preparations of rat cortex or from cells expressing cloned human GABA_{A} receptors.

The present invention also pertains to methods of inhibiting the binding of benzodiazepine compounds, or GABA to the GABA_{A} receptors. Such methods involve contacting a compound provided herein with cells expressing GABA_{A} receptor, wherein the compound is present in an amount sufficient to inhibit benzodiazepine binding or GABA binding to GABA_{A} receptors in vitro. This method includes inhibiting the binding of benzodiazepine compounds to GABA_{A} receptors in vivo (e.g., in a patient given an amount of a compound provided herein that would be sufficient to inhibit the binding of benzodiazepine compounds or GABA to GABA_{A} receptors in vitro). In one embodiment, such methods are useful in treating benzodiazepine drug overdose. The amount of a compound that would be sufficient to inhibit the binding of a benzodiazepine compound to the GABA_{A} receptor may be readily determined via a GABA_{A} receptor binding assay.

Compounds of the invention interact directly with the benzodiazepine site on the GABA_{A} receptor through its ability to inhibit the binding of the selective benzodiazepine ligand [³H]flunitrazepam. Compounds of the invention displace [³H]flunitrazepam binding to rat cortex with an IC50 value of 9.8 nM. This value lies within the range of affinities of established benzodiazepine ligands tested under similar assay conditions (IC50, nM): triazolam (0.5); diazepam (17); zolpidem (26); and flunitrazepam (114). The inhibition of [³ H]flunitrazepam binding to rat cortex membranes by compounds of the invention indicates binding to more than one class of sites (Hill co-efficient=0.6), consistent with the established identity of multiple benzodiazepine receptors. When [³H]flunitrazepam binding to rat cerebellum membranes was examined, compounds of the invention inhibited with an IC50 value of 2 nM and a Hill co-efficient of 0.9. Taken together, these results are consistent with the compound of the invention binding selectively to type I benzodiazepine sites. Type I benzodiazepine ligands have been shown to have a more specific and potent sedative action in animals and in man.

A further index of activity at the benzodiazepine site on the GABA_{A} receptor is the ability to increase the binding of [³⁵S] t-butylbicyclophosphorothionate (TBPS) to rat cortex membranes. TBPS binds to a site on the GABA_{A} receptor which is closely associated with the chloride channel, and an increase in TBPS binding correlates with increased activation of the chloride conductance (i.e., enhanced GABA_{A} receptor function). Compounds which act as agonists at the benzodiazepine site increase TBPS binding to varying degrees, which reflects their relative efficacies. Compound of the invention gave an Eₘₐₓ of 65% (maximum enhancement of TBPS binding over the concentration range employed). This compares with the following values for other benzodiazepine ligands obtained under similar assay conditions (Eₘₐₓ, %): diazepam (55), triazolam (78); flurazepam (71). The value for compounds of the invention predicts potent sedative activity, since it falls within the range of values observed for other benzodiazepine ligands which have sedative properties in animals and man.

Within separate aspects, the present invention provides a variety of in vitro uses for the compounds provided herein. For example, such compounds may be used as probes for the detection and localization of GABA_{A} receptors, in samples such as tissue sections, as positive controls in assays for receptor activity, as standards and reagents for determining the ability of a candidate agent to bind to GABA_{A} receptor, or as radiotracers for positron emission tomography (PET) imaging or for single photon emission computerized tomography (SPECT). Such assays can be used to characterize GABA_{A} receptors in living subjects. Such compounds are also useful as standards and reagents in determining the ability of a potential pharmaceutical to bind to GABA_{A} receptor.

Within methods for determining the presence or absence of GABA_{A} receptor in a sample, a sample may be incubated with a compound as provided herein under conditions that permit binding of the compound to GABA_{A} receptor. The amount of compound bound to GABA_{A} receptor in the sample is then detected. For example, a compound may be labeled using any of a variety of well known techniques (e.g., radiolabeled with a radionuclide such as tritium, as described herein), and incubated with the sample (which may be, for example, a preparation of cultured cells, a tissue preparation or a fraction thereof). A suitable incubation time may generally be determined by assaying the level of binding that occurs over a period of time. Following incubation, unbound compound is removed, and bound compound detected using any method for the label employed (e.g., autoradiography or scintillation counting for radiolabeled compounds; spectroscopic methods may be used to detect luminescent groups and fluorescent groups). As a control, a matched sample may be simultaneously contacted with radiolabeled compound and a greater amount of unlabeled compound. Unbound labeled and unlabeled compound is then removed in the same fashion, and bound label is detected. A greater amount of detectable label in the test sample than in the control indicates the presence of capsaicin receptor in the sample. Detection assays, including receptor autoradiography (receptor mapping) of GABA_{A} receptors in cultured cells or tissue samples may be performed as described by Kuhar in sections 8.1.1 to 8.1.9 of Current Protocols in Pharmacology (1998) John Wiley & Sons, New York.

Compounds provided herein may also be used within a variety of well known cell culture and cell separation methods. For example, compounds may be linked to the interior surface of a tissue culture plate or other cell culture support, for use in immobilizing GABA_{A} receptor-expressing cells for screens, assays and growth in culture. Such linkage may be performed by any suitable technique, such as the methods described above, as well as other standard techniques. Compounds may also be used to facilitate cell identification and sorting in vitro, permitting the selection of cells expressing a GABA_{A} receptor. Preferably, the compound(s) for use in such methods are labeled as described herein. Within one preferred embodiment, a compound linked to a fluorescent marker, such as fluorescein, is contacted with the cells, which are then analyzed by fluorescence activated cell sorting (FACS).

Within other aspects, methods are provided for modulating binding of ligand to a GABA_{A} receptor in vitro or in vivo, comprising contacting a GABA_{A} receptor with a sufficient amount of a compound provided herein, under conditions suitable for binding of ligand to the receptor. The GABA_{A} receptor may be present in solution, in a cultured or isolated cell preparation or within a patient. Preferably, the GABA_{A} receptor is a present in the brain of a mammal. In general, the amount of compound contacted with the receptor should be sufficient to modulate ligand binding to GABA_{A} receptor in vitro.

Also provided herein are methods for altering the signal-transducing activity of cellular GABA_{A} receptor (particularly the chloride ion conductance), by contacting GABA_{A} receptor, either in vitro or in vivo, with a sufficient amount of a compound as described above, under conditions suitable for binding of ligand to the receptor. The GABA_{A} receptor may be present in solution, in a cultured or isolated cell preparation or within a patient. In general, the amount of compound contacted with the receptor should be sufficient to modulate ligand binding to GABA_{A} receptor in vitro. An effect on signal-transducing activity may be assessed as an alteration in the electrophysiology of the cells, using standard techniques. If the receptor is present in an animal, an alteration in the electrophysiology of the cell may be detected as a change in the animal's feeding behavior. The amount of a compound that would be sufficient to alter the signal-transducing activity of GABA_{A} receptors may be determined via a GABA_{A} receptor signal transduction assay. The cells expressing the GABA receptors in vivo may be, but are not limited to, neuronal cells or brain cells. Such cells may be contacted with compounds of the invention through contact with a body fluid containing the compound, for example through contact with cerebrospinal fluid. Alteration of the signal-transducing activity of GABA_{A} receptors in vitro may be determined from a detectable change in the electrophysiology of cells expressing GABA_{A} receptors, when such cells are contacted with a compound of the invention in the presence of GABA_{A}.

Combination administration can be carried out in a fashion analogous to that disclosed in Da-Rocha, et al., J. Psychopharmacology (1997) 11(3) 211 218; Smith, et al., Am. J. Psychiatry (1998) 155(10) 1339 45; or Le, et al., Alcohol and Alcoholism (1996) 31 Suppl. 127 132. See also the discussion of the use of the GABA_{A} receptor ligand 3-(5-methylisoxazol-3-yl)-6-(1-methyl- 1,2,3-triazol-4-yl)methyloxy- 1,2,4-triazolo[3,4a]phthalazine in combination with nicotinic agonists and muscarinic agonists. In addition, WO 99/37303 describes the use of a class of GABA_{A} receptor ligands, 1,2,4-triazolo[4,3-b]pyridazines, in combination with SSRIs.

The term “Restorative Sleep” means sleep which produces a rested state upon waking; the term “Sleep Disorder” means Insomnia and Obstructive Sleep Apnea; the term “Insomnia” means Primary Insomnia, Insomnia related to another Mental Disorder, and Substance-Induced Insomnia; the term “Primary Insomnia” means difficulty in initiating sleep, in maintaining sleep or having restorative sleep which is not caused by a Mental Disorder or due to physiological effects of taking or withdrawing from certain substances (substance-induced). As used herein, it also includes Circadian Rhythm Insomnia which is insomnia due to a change in the normal sleep-wake schedule (shift changes, jet lag, etc.); the term “Insomnia related to another Mental Disorder” means difficulty in initiating sleep, in maintaining sleep or having restorative sleep which is caused by an underlying Mental Disorder such as, for example, depression, anxiety or schizophrenia; the term “Substance-Induced Insomnia” means difficulty in initiating sleep, in maintaining sleep or having restorative sleep which is caused by physiological effects of taking or withdrawing from certain substances such as caffeine, alcohol, amphetamine, opioids, sedatives, hypnotics and anxiolytics; and the term “Obstructive Sleep Apnea” means repeated episodes of upper-airway obstruction during sleep and is normally characterized by loud snores or brief gasps that alternate with episodes of silence.

### Diagnostic Methods and Kits

According to another embodiment, the invention provides a method of determining the concentration of indiplon in a solution or a biological sample, said method comprising the steps of:

a) adding a known concentration of a compound of formula I above, wherein: R¹ is thienyl; and R² is hydrogen; to the biological sample;

b) subjecting the solution or biological sample to a measuring device that distinguishes indiplon from the compound of formula I;

c) calibrating the measuring device to correlate the detected quantity of the compound of formula I with the known concentration of the compound of formula I added to the biological sample or solution;

d) measuring the quantity of indiplon in the biological sample with said calibrated measuring device; and

e) determining the concentration of indiplon in the solution of sample using the correlation between detected quantity and concentration obtained for a compound of formula I.

According to another embodiment, the invention provides a method of determining the concentration of zaleplon in a solution or a biological sample, said method comprising the steps of:

a) adding a known concentration of a compound of formula IV, wherein: R² is hydrogen and Z² is hydrogen; to the biological sample;

b) subjecting the solution or biological sample to a measuring device that distinguishes zaleplon from the compound of formula IV;

c) calibrating the measuring device to correlate the detected quantity of the compound of formula IV with the known concentration of the compound of formula IV added to the biological sample or solution;

d) measuring the quantity of zaleplon in the biological sample with said calibrated measuring device; and

e) determining the concentration of zaleplon in the solution of sample using the correlation between detected quantity and concentration obtained for a compound of formula IV.

Measuring devices that can distinguish indiplon from a compound of formula I or zaleplon from a compound of formula IV include any measuring device that can distinguish between two compounds that are of identical structure except that one contains one or more heavy atom isotope versus the other (i.e., that differ from one another only in isotopic abundance). Exemplary measuring devices include a mass spectrometer, NMR spectrometer, or IR spectrometer.

In another embodiment, the invention provides a method of evaluating the metabolic stability of a compound of any one of formulae I, II or IV, comprising the steps of contacting the compound of formula I or its acid addition salt with a metabolizing enzyme source for a period of time; and comparing the amount of said compound and metabolic products of said compounds after said period of time.

In a related embodiment, the invention provides a kit comprising, in separate vessels: a) a non-deuterated compound; and b) a metabolizing enzyme source. The kit is useful for comparing the metabolic stability of a compound of formula I or IV with its non-deuterated compound, as well as evaluating the effect of deuterium and ¹³C replacement at various positions on a compound of formula I or IV. In a preferred embodiment, the kit further comprises instructions for using the non-deuterated compound and said metabolizing enzyme source to evaluate the metabolic stability of a compound of formula I or IV.

In a related embodiment, the invention provides a method of evaluating the metabolic stability of a compound of formula I or IV in a patient following administration of the compound of formula I or IV. This method comprises the steps of obtaining a serum, urine or feces sample from the patient at a period of time following the administration of the compound of formula I or IV to the subject; and comparing the amount of the compound of formula I or IV with the metabolic products of the compound of formula I or IV in the serum, urine or feces sample.

The present invention also provides kits for use to treat insonmia. These kits comprise (a) a pharmaceutical composition comprising a compound of formula I or IV or a salt, hydrate, or solvate thereof, wherein said pharmaceutical composition is in a container; and (b) instructions describing a method of using the pharmaceutical composition to treat insomnia.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In one embodiment, the container is a blister pack.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

In certain embodiment, the kits of this invention may comprise in a separate vessel of container a pharmaceutical composition comprising a second therapeutic agent, such as one of those listed above for use for co-administration with a compound of this invention.

In order that the invention might be more fully understood, the following examples are set forth. They are not intended to limit the scope of the invention and further examples will be evident to those of ordinary skill in the art. In each example set forth herein, carbon shall be ¹²C, and hydrogen shall by ¹H, each incorporated at its natural abundance, unless otherwise specified.

### EXAMPLES

In the chemical synthesis examples set forth below and asterisk (*) indicates optional deuteration of the indicated compound. In certain examples the location of the deuterium atoms in the compound are specified.

### Example 1

### Synthesis of N-d₃-Methyl-N-{3-[3-(thiophene-2-carbonyl)-pyrazolo[1,5-a]pyrimidin-7-yl]-phenyl}-acetamide (Compound 1)

β-Dimethylamino-1-(2-thienyl)-2-propen-1-one (B). A mixture of 2-acetylthiophene (80 mmol) and dimethylformamide dimethyl acetal (126 mmol) is heated under reflux under nitrogen for 3 hrs. The reaction mixture is cooled and hexane (300 mL) added. The solid is collected by filtration to yield product B.

5-(2-Thienyl)isoxazole (C). A mixture of B (100 mmol) and hydroxylamine hydrochloride (101 mmol) in anhydrous methanol (100 mL) is refluxed under nitrogen for 2 hrs. The reaction mixture is cooled, concentrated and partitioned between water and dichloromethane. The dichloromethane layer is dried with anhydrous sodium sulfate, filtered and concentrated to yield product C.

α-[(Dimethylamino)methylene]-β-oxo-2-thiophenepropanenitrile (D). A mixture of C (86 mmol) and dimethylformamide dimethyl acetal (188 mmol) is refluxed under nitrogen for 8 hrs. Solid precipitates from the reaction mixture. Hexane is added to the reaction mixture and the solid is collected by filtration, giving product D.

(3-Amino-1H-pyrazol-4-yl)-2-thienylmethanone (F). To a mixture of 2-[(dimethylamino)methylene]-3-oxo-3-(2-thienyl)propane nitrile (E, 20.6 g, 100 mmol) and aminoguanidine nitrate (17.1 g, 125 mmol) in ethanol (120 mL) was added a solution of sodium hydroxide (5 g, 125 mmol) in water (12.5 mL). The reaction mixture was refluxed 6 hours and then the volatiles were removed. The crude solid was taken up in water (250 mL). The precipitate was collected by suction filtration, washed with water, and dried at 60° C. for 5 hours to give F (13.7 g) as a yellow solid.

N-[3-(3-(Dimethylamino-acryloyl)-phenyl]-acetamide (H). A solution of 3′-acetomidoacetophenone (G; 100 g, 564.3 mmol) in N,N-dimethylformamide dimethyl acetal (166 mL, 1.25 mol) was refluxed in a 1-L round bottom flask equipped with a Dean-Stark apparatus for 2 hours. The volatiles were removed under reduced pressure and the solid were triturated with MTBE (500 mL) to give H (126 g, 96%) as an orange solid.

N-[3-(3-(Dimethylamino-acryloyl)-phenyl]-N-d₃-methylacetamide (*J). To a solution of H (46.4 g, 200 mmol) in anhydrous N,N-dimethylformamide (240 mL) at 0° C. was added sodium hydride (10.0 g, 60 wt % in mineral oil, 250 mmol) in portions. After the evolution of gas, a solution of iodomethane-d₃ (13.1 mL, 210 mmol) in DMF (100 mL) was added dropwise. The reaction was allowed to warm to room temperature and stirred overnight. Then the reaction was cooled back to 0° C. and slowly quenched with water (500 mL). The reaction mixture was extracted with DCM (3×600 mL). The combined organic layer were washed with brine (2×400 mL), dried over sodium sulfate, and evaporated in vacuo to give a crude solid that was triturated with MTBE (400 mL) to give *J (36 g) as a yellow solid.

N-d₃-Methyl-N-{3-[3-(thiophene-2-carbonyl)-pyrazolo[1,5-a]pyrimidin-7-yl]-phenyl}-acetamide (Compound 1). To a suspension of J (3.74 g, 15 mmol) in acetic acid (150 mL) was added F (2.90 g, 15 mmol). The suspension was refluxed for 8 hours and the volatiles were removed under reduced pressure. The crude solid was dissolved in DCM (50 mL), precipitated with heptane (200 mL), and filtered to give a yellow solid that was chased with ethanol (3×100 mL) and water (3×100 mL) to yield Compound 1 (3.41 g). The structure was confirmed by each of ¹H-NMR Spectrum analysis (DMSO-d₆), COSY Spectrum analysis (DMSO-d₆), ¹³C-NMR Spectrum analysis (DMSO-d₆), IR (KBr pellet), Mass Spectrum analysis and Elemental analysis. Mass spec: (M+H)⁺ Calc: 380.45; Found: 380.0. Compound 1 was found to have a molecular weight of 379.45, a melting point of 189.9-192.1° C. and was >95% pure by HPLC analysis (ZORBAX 4.6×50 mm column SB-Aq 3.5 μm; retention time=5.722 min; mobile phase:CAN/water/formic acid; wavelength=210 nm). ¹H-NMR (300 MHz, DMSO-d₆): δ1.91 (bs, 3H), 7.30 (dd, J₁=5.0 Hz, J₂=3.8 Hz, 1H), 7.58-7.713 (m, 3H), 8.04-8.09 (m, 3H), 8.22 (dd, J₁=3.8 Hz, J₂=1.2 Hz, 1H), 8.84 (s, 1H), 8.91 (d, J=4.4 Hz, 1H). ¹³C-NMR (75 MHz, DMSO-d₆): δ23.14, 110.73, 111.10, 129.26, 130.46, 134.99, 135.10, 145.02, 145.30, 146.85, 147.81, 148.45, 153.63, 179.33. HPLC (method: ZORBAX 4.6×50 mm SB-Aq 3.5 μm column; mobile phase: 2-98% ACN+0.1% formic acid for 6 min with MSD in ESI positive mode, 0.63 mL/min; Wavelength: 210 nm): retention time: 5.722 min. MS (M+H⁺): 380.0. Elemental Analysis (C₂₀H₁₃D₃N₄O₂S.0.4 H₂O): Calculated: C=62.61, H=4.41, N=14.60, S=8.36. Found: C=62.28, H=4.05, N=14.57, S=8.31.

### Example 2

### Compounds of the Invention in Accordance with Scheme 3

4-bromo-5-aminopyrazole (M). 5-aminopyrazole (L) (200 mmol) is dissolved in glacial acetic acid. Br₂ (400 mmol) is added and the reaction is stirred for 1 hr. The reaction mixture is extracted with ethyl acetate, washed three times with saturated sodium bicarbonate solution, and the organic layer is concentrated to provide M.

3-bromopyrazolo[1,5-a]pyrimidine derivative (*N). To a stirring solution of M (100 mmol) in glacial acetic acid is added *N-[3-[3-(dimethylamino)-2-propenoyl]phenyl]-N-methylacetamide (*J) (95 mmol). The reaction mixture is refluxed for 12 hr, extracted into ethyl acetate, and washed with saturated sodium bicarbonate solution, to afford *N.

N-Methyl-N-[3-[3-(2-thienylcarbonyl)-pyrazolo[1.5-a]pyrimidin-7-yl]phenyl]acetamide (*K). To a mixture of *N (10 mmol) and 2-thienylboronic acid (P) (10 mmol) in toluene is added Pd(PPh₃)₄ and a stream of CO. The reaction mixture is stirred for 24 hr. The reaction mixture is filtered through celite, extracted into ethyl acetate, and washed with saturated sodium bicarbonate solution. Evaporation of all volatiles is carried out on a rotary evaporator and hexane is added until crystallization is observed. The mixture is cooled and filtered to give deuterated compound *K.

### Example 3

### Compounds of the Invention in Accordance with Scheme 4

Pyrazolo[1,5-a]pyrimidine derivative (*Q). To a stirring solution of L (150 mmol) in acetic acid is added *J (150 mmol). The reaction is heated to reflux for 12 hr, extracted into ethyl acetate, and washed with saturated sodium bicarbonate solution. The organic layers are concentrated under vacuum to provide *Q.

N-Methyl-N-[3-[3-(2-thienylcarbonyl)-pyrazolo[1.5-a]pyrimidin-7-yl]phenyl]acetamide (*K). To a solution of *Q (50 mmol) in ether at 0° C. is added 2-thienylcarbonyl chloride (R) (50 mmol) and AlCl₃ (3 mmol). The reaction is stirred for 4 hr, extracted into ether, washed with water, and concentrated. Hexane is added until crystallization is observed. The mixture is cooled and filtered to give deuterated compound *K.

### Example 4

### Compounds of the Invention in Accordance with Scheme 5

5-aminopyrazole-4-carbonitrile (T). To a solution of ethoxymethylenemalonodinitrile (S) (80 mmol) in benzene is added hydrazine (150 mmol). The reaction is stirred at reflux for 1 hr, diluted into ethyl acetate, washed with sodium bicarbonate solution and concentrated under vacuum, to provide T.

3-cyanopyrazolo[1,5-a]pyrimidine derivative (*U). T (50 mmol) is dissolved in acetic acid, and *J (50 mmol) is added. The reaction is heated to reflux, extracted into ethyl acetate, washed with sodium bicarbonate solution and concentrated under vacuum to yield *U.

N-Methyl-N-[3-[3-(2-thienylcarbonyl)-pyrazolo[1.5-a]pyrimidin-7-yl]phenyl]acetamide (*K). To a solution of *U (30 mmol) in ether at 0° C. is added 2-bromothiophene (V) (30 mmol) and Mg shot (10 mmol). The reaction is stirred, extracted into ether, washed with water, and concentrated. Hexane is added until crystallization is observed. The mixture is cooled and filtered to give deuterated compound *K.

### Example 5

### Compounds of the Invention in Accordance with Scheme 6

7-hydroxypyrazolo[1,5-a]pyrimidine-3-carboxylic acid ethyl ester (Y). To a solution of 5-aminopyrazole-4-carboxylic acid ethyl ester (W) (100 mmol) in acetic acid is added either, 3,3-diethoxypropionic acid ethyl ester or 3-oxopropionic acid ethyl ester. The reaction is heated under reflux for 2 hr, extracted into ether, washed with water, and concentrated to provide Y.

7-chloropyrazolo[1,5-a]pyrimidine-3-carboxylic acid ethyl ester (Z). Y (25 mmol) is treated with POCl₃ (50 mmol) at 0° C. in ether. The reaction is stirred for 1 hr, diluted in ether, washed with sodium chloride solution, and concentrated to yield the corresponding chloro derivative Z.

Pyrazolo[1,5-a]pyrimidine-carboxylic acid ethyl ester (*BB). Compound Z (10 mmol) in a solution of ethanol with *3-(N-methylacetamido) phenylboronic acid (AA) (10 mmol), Pd(PPh₃)₄ (2 mmol) and Na₂CO₃ (50 mmol) is heated at reflux for 12 hr. Extraction into ethyl acetate, washing with sodium chloride solution, and concentration affords *BB.

N-Methyl-N-[3-[3-(2-thienylcarbonyl)-pyrazolo[1.5-a]pyrimidin-7-yl]phenyl]acetamide (*K). To a solution of *BB (30 mmol) in ether at 0° C. is added V (30 mmol) and Mg shot (10 mmol). The reaction is stirred, extracted into ether, washed with water, and concentrated. Hexane is added until crystallization is observed. The mixture is cooled and filtered to give deuterated compound *K.

### Example 6

The sedative effects of compounds of the invention are measured in rats using a panel of standard tests which monitor the effects of drugs on motor activity, muscle relaxation and motor coordination. The ED₅₀ for a decrease in motor activity is measured upon dosing. The ED₅₀ for the muscle relaxant effects of compounds of the invention are measured by the inclined screen grip strength test. Similarly, the ED₅₀ is noted for the rod walking test, which measures coordinated motor ability.

Compounds of the invention increase locomotor activity and increase positive results in the inclined screen test.

Compounds of the invention produce dose-related increases in sleep duration in rats. EEG studies in squirrel monkeys are used to indicate that compounds of the invention cause changes in the EEG power spectrum, which changes can seen for benzodiazepine sedative-hypnotic agents.

### Example 7

Vigilance tests based on reaction time measurements in monkeys measure drug induced slowing of responses and decreases in attentional or cognitive processes. A compound of the invention is delivered orally one hour before testing and monitored for producing dose related decrements in both the accuracy and latency of the responses.

The Thirsty Rat conflict Procedure involves rats which are deprived of water for 48 hours. They are then given brief electrical shocks following each 20th lick of a drinking tube. These shocks create a “conflict situation” which results in a marked decrease in licking (drinking) behavior. Oral dosing with a compound of the invention is monitored for an increase in the number of shocks accepted by the treated rats compared to rats in the control groups which received only the dosing vehicle.

The Squirrel Monkey Conflict Procedure involves monkeys which are initially trained to press a bar to receive a food reward. In later sessions the animals are given occasional electrical shocks following randomly selected bar presses. These animals respond to the conflict situation by substantially lowering their levels of bar-pressing activity.

### Example 8

Benzodiazepine sedative-hypnotic agents are potent anticonvulsants in animals, and are used clinically for the treatment of status epilepticus. Oral doses of compounds of the invention are monitored for effectiveness in blocking convulsions in rats which resulted from injections of agents known to induce convulsion. In addition, orally administered compounds of the invention are monitored for effectiveness in blocking convulsions induced by a 150 mA, 60 Hz electrical shock delivered transcorneally for 0.3 seconds. The anticonvulsant potency of compounds of the invention is determined by the methods described herein.

### Example 9

In order to assess tolerance to compounds of the invention, its effects are tested on rat motor activity, and on grip strength using the inclined screen procedures after single and repeated administrations. Animals given daily treatment of a compound of the invention for four days are monitored for an increase in the ED₅₀ when tested for motor activity, and when tested for grip strength on the fourth day of dosing.

### Example 10

Human Microsomal Assay: The metabolic stability of the present compounds may be evaluated in one or more microsomal assays that are known in the art. See, for example, Obach, R. S. Drug Metab Disp 1999, 27, p. 1350 “Prediction of human clearance of twenty-nine drugs from hepatic microsomal intrinsic clearance data: An examination of in vitro half-life approach and nonspecific binding to microsomes”; Houston, J. B. et al., Drug Metab Rev 1997, 29, p. 891 “Prediction of hepatic clearance from microsomes, hepatocytes, and liver slices”; Houston, J. B. Biochem Pharmacol 1994, 47, p. 1469 “Utility of in vitro drug metabolism data in predicting in vivo metabolic clearance”; Iwatsubo, T et al., Pharmacol Ther 1997, 73, p. 147 “Prediction of in vivo drug metabolism in the human liver from in vitro metabolism data”; and Lave, T. et al., Pharm Res 1997, 14, p. 152 “The use of human hepatocytes to select compounds based on their expected hepatic extraction ratios in humans”; each of which are incorporated herein in their entirety.

The objectives of this study were to determine the metabolic stability of the test compounds in pooled liver microsomal incubations and to perform full scan LC-MS analysis for the detection of major metabolites. Samples of the test compounds, exposed to pooled human liver microsomes, were analyzed using HPLC-MS (or MS/MS) detection. For determining metabolic stability, multiple reaction monitoring (MRM) was used to measure the disappearance of the test compounds. For metabolite detection, Q1 full scans were used as survey scans to detect the major metabolites.

Experimental Procedures: Human liver microsomes were obtained from Absorption Systems L.P. (Exton, Pa.). Details about the matrices used in the experiments are shown below. The incubation mixture was prepared as follows:

Reaction Mixture Composition

| | | |
|---|---|---|
| Liver Microsomes | 1.0 | mg/mL |
| NADPH | 1 | mM |
| Potassium Phosphate, pH 7.4 | 100 | mM |
| Magnesium Chloride | 10 | mM |
| Test Compound (Indiplon or Compound 1) | 1 | μM |

Incubation of Test Compounds with Liver Microsomes: The reaction mixture, minus cofactors, was prepared. An aliquot of the reaction mixture (without cofactors) was incubated in a shaking water bath at 37° C. for 3 minutes. Another aliquot of the reaction mixture was prepared as the negative control. The test compound was added into both the reaction mixture and the negative control at a final concentration of 1 μM. An aliquot of the reaction mixture was prepared as a blank control, by the addition of plain organic solvent (not the test compound). The reaction was initiated by the addition of cofactors (not into the negative controls), and then incubated in a shaking water bath at 37° C. Aliquots (200 μL) were withdrawn in triplicate at 0, 15, 30, 60, and 120 minutes and combined with 800 μL of ice-cold 50/50 acetonitrile/dH₂O to terminate the reaction. The positive controls, testosterone and propranolol, were run simultaneously with the test compounds in separate reactions.

All samples were analyzed using LC-MS (or MS/MS). An LC-MRM-MS/MS method was used for metabolic stability. Also, Q1 full scan LC-MS methods were performed on the blank matrix and the test compound incubation samples. The Q1 scans served as survey scans to identify any sample unique peaks that might represent the possible metabolites. The masses of these potential metabolites can be determined from the Q1 scans.

Results: Metabolic Stability: Compound 1 was prepared and tested in the human liver microsome assay described above along with indiplon. After 60 and 120 minutes of exposure in the microsomal assay, Compound 1 was more resistant to microsomal degradation than indiplon. For Compound 1, 66% was remaining after 60 minutes of exposure and 59% was remaining after 120 minutes. By contrast, 50% and 42% of the indiplon was remaining after 60 and 120 minutes, respectively. The half life of Compound 1 was calculated to be 74.4 minutes. The half-life of Indiplon was 59.0 minutes. The ratio of half lives of Compound 1 to Indiplon was calculated to be 1.261. These results indicate that the deuterium substitution in Compound 1 was effective in slowing the cytochrome-mediated oxidation.

Co-incubation of Compound 1 and Indiplon with Human Liver Microsomes: In another set of experiments Compound 1 and Indiplon were incubated together with human liver microsomes and their metabolism followed over time to eliminate any potential variability in samples. The reaction mixture compositions utilized in these experiments were as follows:

Reaction Mixture Composition A:

| | | |
|---|---|---|
| Liver Microsomes | 1.0 | mg/mL |
| NADPH | 2 | mM |
| Potassium Phosphate, pH 7.4 | 100 | mM |
| Magnesium Chloride | 3 | mM |
| Compound 1 | 0.5 | μM |
| Indiplon | 0.5 | μM |

Reaction Mixture Composition B:

| | | |
|---|---|---|
| Liver Microsomes | 1.0 | mg/mL |
| NADPH | 2 | mM |
| Potassium Phosphate, pH 7.4 | 100 | mM |
| Magnesium Chloride | 3 | mM |
| Compound 1 | 0.1 | μM |
| Indiplon | 0.1 | μM |

Reaction Mixture Composition C:

| | | |
|---|---|---|
| Liver Microsomes | 2.0 | mg/mL |
| NADPH | 2 | mM |
| Potassium Phosphate, pH 7.4 | 100 | mM |
| Magnesium Chloride | 3 | mM |
| Compound 1 | 0.2 | μM |
| Indiplon | 0.2 | μM |

HPLC-grade solvents were used for preparation of all solutions. Dosing solutions of test agents were initially prepared at five times the final concentrations of the various reaction mixtures indicated above. Dosing solutions were warmed to 37° C. prior to the start of the assay. Two sets of triplicate wells were prepared in a 96-well deep well plate for each reaction mixture and warmed to 37° C. At the start of the assay, dosing solutions were diluted five-fold into the reaction wells. The reaction plate was incubated at 37° C., and 30 μL aliquots removed immediately after start (15 sec), and after each of 10 min, 20 min, 30 min, 40 min and 60 min of reaction. The aliquots were added to 30 μL of ice cold STOP Solution (acetonitrile containing 0.3% acetic acid with 3 mM haloperidol as an internal standard) in a separate 96-well plate. The stopped reactions were incubated at least 10 min at −20° C., and 30 μL of water was added. The plates were centrifuged for 15 min at 2000 rpm, 4° C. Approximate 60 μL of the supernatant was removed without disturbing the pellet and transferred to a fresh 96-well plate for analysis. Analysis samples were stored at −20° C. and analyzed by LC-MS.

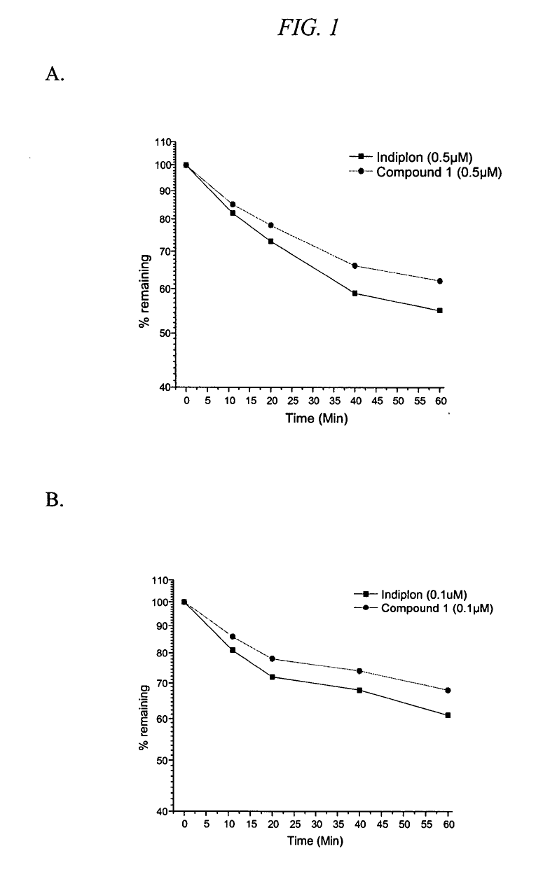

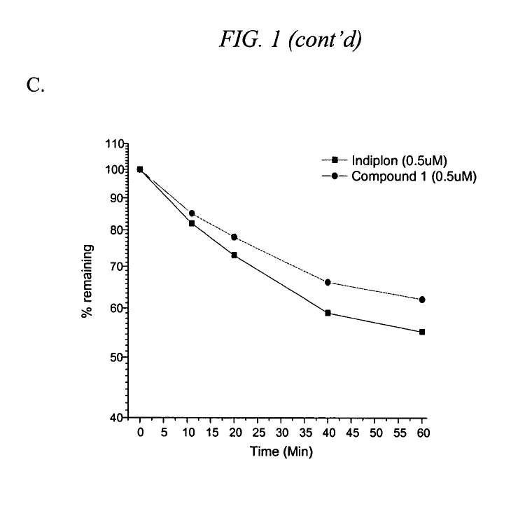

FIG. 1, panel A, demonstrates that metabolism of Compound 1 at concentration of 0.5 μM in a 1.0 mg/ml human microsome preparation is significantly slower than metabolism of the same concentration of Indiplon in the same preparation. The half-life for Compound 1 was calculated to be 204.3 minutes, while the half-life for Indiplon was 162.2 minutes. The ratio of half lives of Compound 1 to Indiplon was calculated to be 1.260.

FIG. 1, panel B, demonstrates that metabolism of Compound 1 at concentration of 0.1 μM in a 1.0 mg/ml human microsome preparation is significantly slower than metabolism of the same concentration of Indiplon in the same preparation. The half-life for Compound 1 was calculated to be 273.4 minutes, while the half-life for Indiplon was 217.5 minutes. The ratio of half lives of Compound 1 to Indiplon was calculated to be 1.257.

FIG. 1, panel C, demonstrates that metabolism of Compound 1 at concentration of 0.2 μM in a 2.0 mg/ml human microsome preparation is significantly slower than metabolism of the same concentration of Indiplon in the same preparation. The half-life for Compound 1 was calculated to be 152.6 minutes, while the half-life for Indiplon was 116.3 minutes. The ratio of half lives of Compound 1 to Indiplon was calculated to be 1.312.

These results confirm that the deuterated compounds of the present invention are more resistant to cytochrome P450 oxidation than Indiplon and thus may have an advantageously longer lasting effect when administered to human subjects.

### Example 10

Benzodiazepine Receptor Binding. Compound 1 and Indiplon were compared in their ability to bind to compete with [³H] flunitrazepam (0.4 nM) for binding to the benzodiazepine receptor of the rat cerebral cortex using the method of Speth, R C et al., 1979, Life Sci., 24:351-358. Varying concentrations of Indiplon or Compound 1 (30 pM-1 μM) were used. Diazepam (3 μM) was used as a non-specific receptor binding compound. Binding was allowed to proceed at 4° C. for 60 minutes. Quantification of binding was determined by scintillation counting.

Using the above-described assay Indiplon was determined to have an IC₅₀ of 3.6×10⁻⁹ M and a Kᵢ of 3×10⁻⁹ M. Compound 1 had an IC₅₀ of 2.9×10⁻⁹ M and a Kᵢ of 2.4×10⁻⁹ M. These results show that Compound 1 binds specifically to the benzodiazepine receptor in rat cerebral cortex and that such binding is essentially identical to that of Indiplon.

All of the features, specific embodiments and particular substituents disclosed herein may be combined in any combination. Each feature, embodiment or substituent disclosed in this specification may be replaced by an alternative feature, embodiment or substituent serving the same, equivalent, or similar purpose. In the case of chemical compounds, specific values can be combined in any combination resulting in a stable structure. Furthermore, specific values (whether preferred or not) for substituents in one type of chemical structure may be combined with values for other substituents (whether preferred or not) in the same or different type of chemical structure. Thus, unless expressly stated otherwise, each feature, embodiment or substituent disclosed is only an example of a generic series of equivalent or similar features feature, embodiments or substituents.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

The contents of all references (including literature references, issued patents, published patent applications, and co-pending patent applications) cited throughout this application are hereby expressly incorporated herein in their entireties by reference.

We claim:

1. A compound of Formula I: 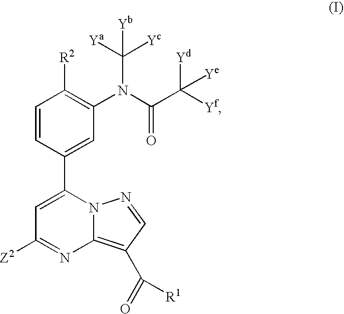
or a pharmaceutically acceptable salt, hydrate, or solvate of said compound, wherein:
each of Y^{a}, Y^{b}, Y^{c}, Y^{d}, Y^{e}, and Y^{f} is independently selected from hydrogen and deuterium, wherein at least one of Y^{a}, Y^{b}, Y^{c}, Y^{d}, Y^{e}, and Y^{f} is deuterium;
R¹ is selected from phenyl optionally substituted with 1 to 2 substituents independently selected from halogen, (C₁-C₃)alkoxy, (C₁-C₃)alkyl, (C₁-C₃)alkylamino, (C₁-C₃)dialkylamino, methylenedioxy, (C₁-C₃)alkylsulfonyl, and (C₁-C₃)alkanoylamino; naphthalenyl; furanyl; thiazolyl; biphenyl; thienyl; and pyridinyl, wherein each thiazolyl, biphenyl, thienyl, or pyridinyl is optionally substituted with 1 or 2 substitutents independently selected from halogen, (C₁-C₃)alkoxy and (C₁-C₃)alkyl;
R² is selected from hydrogen, halogen, (C₁-C₃)alkoxy and (C₁-C₃)alkyl; and
Z² is selected from hydrogen and deuterium.

2. The compound of claim 1, wherein at least one of Y^{a}, Y^{b}, Y^{c} is deuterium.

3. The compound of claim 2, wherein Y^{a}, Y^{b}, and Y^{c} are simultaneously deuterium.

4. The compound of any one of claims 1 to 3, wherein at least one of Y^{d}, Y^{e}, and Y^{f} is deuterium.

5. The compound of claim 1, wherein at least two of Y^{a}, Y^{b}, Y^{c}, Y^{d}, Y^{e}, and Y^{f} are deuterium.

6. The compound of any one of claims 1 to 5, wherein R¹ is thienyl.

7. The compound of claim 1 having the structure: 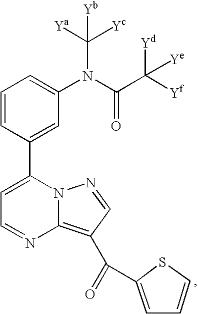
and selected from one of the compounds listed in the table below:

| Compound | Y^{a} | Y^{b} | Y^{c} | Y^{d} | Y^{e} | Y^{f} |
|---|---|---|---|---|---|---|
| 1 | D | D | D | H | H | H |
| 2 | D | D | D | D | H | H |
| 3 | D | D | D | D | D | H |
| 4 | D | D | D | D | D | D |

, or a pharmaceutically acceptable salt, hydrate, or solvate of any of the above compounds.

8. A compound of Formula II: 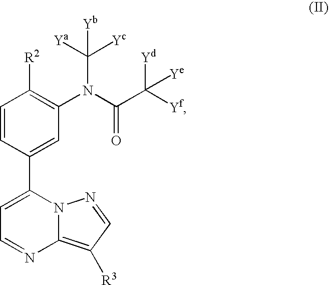
or a pharmaceutically acceptable salt, hydrate, or solvate of pound, wherein:
each of Y^{c}, Y^{b}, Y^{c}, Y^{d}, Y^{e}, and Y^{f} is independently selected from hydrogen and deuterium, wherein at least one of Y^{a}, Y^{b}, Y^{c}, Y^{d}, Y^{e}, and Y^{f} is deuterium;
R² is selected from hydrogen, halogen, (C₁-C₃)alkoxy and (C₁-C₃)alkyl; and
R³ is selected from hydrogen, cyano, nitro, azido, halogen, and C(O)OR⁴, wherein R⁴ is hydrogen, (C₁-C₃)alkyl, or (C₆-C₂₀)aryl; and
n is an integer from 0 to 2.

9. The compound of claim 8, wherein R³ is hydrogen.

10. The compound of claim 8, wherein R³ is cyano.

11. The compound of claim 8, wherein:
R³ is C(O)OR⁴; and
R⁴ is (C₁-C₃)alkyl.

12. A compound of Formula III: 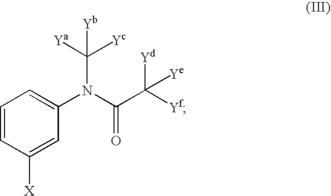
or a pharmaceutically acceptable salt, hydrate, or solvate of said compound, wherein:
each of Y^{a}, Y^{b}, Y^{e}, Y^{d}, Y^{e}, and Y^{f} is independently selected from hydrogen and deuterium, wherein at least one of Y^{a}, Y^{b}, Y^{c}, Y^{d}, Y^{e}, and Y^{f} is deuterium;
X is selected from B(OR⁵)₂, C(O)(CH═CH)ₙN(R⁵)(R⁶), cyano, nitro, azido, and halogen;
each R⁵ is independently selected from hydrogen and (C₁-C₃)alkyl;
R⁶ is selected from hydrogen, (C₁-C₃)alkyl, and (C₆-C₂₀)aryl; and
n is an integer from 0 to 2.

13. The compound of claim 12, wherein X is B(OR⁵)₂.

14. The compound of claim 13, wherein R⁵ is H.

15. The compound of claim 12, wherein X is C(O)(CH═CH)ₙN(R⁵)(R⁶).

16. The compound of claim 15, wherein n is 1.

17. The compound of claim 15 or 16, wherein:
R⁵ is (C₁-C₃)alkyl; and
R⁶ is (C₁-C₃)alkyl.

18. The compound of claim 8 or 12, selected from one of the following compounds: 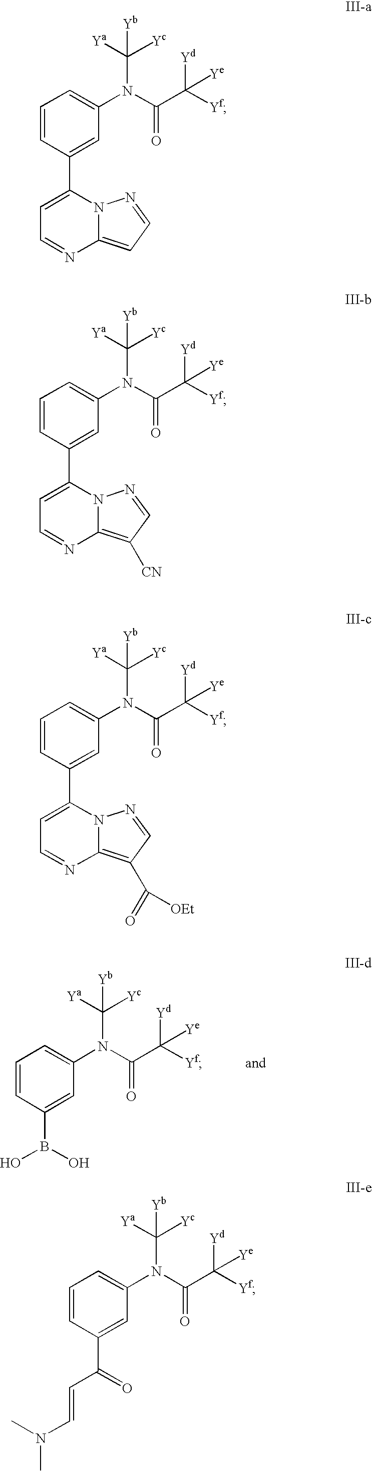
wherein Y^{a}, Y^{b}, and Y^{c} are simultaneously deuterium; and each of Y^{d}, Y^{e}, and Y^{f} are independently selected from hydrogen and deuterium.

19. A compound of Formula IV: 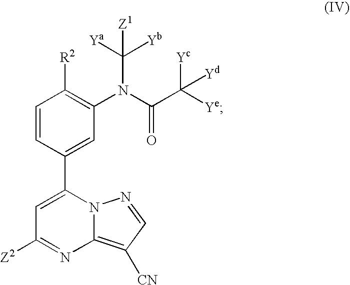
or a pharmaceutically acceptable salt, hydrate, or solvate of said compound, wherein:
each of Y^{a}, Y^{b}, Y^{c}, Y^{d}, and Y^{e} is independently selected from hydrogen and deuterium, wherein at least one of Y^{a}, Y^{b}, Y^{c}, Y^{d}, and Y^{e} is deuterium;
R² is selected from hydrogen, halogen, (C₁-C₃)alkoxy and (C₁-C₃)alkyl;
Z¹ is selected from hydrogen, deuterium, —CH₃; —CH₂D; —CHD₂; and —CD₃; and
Z² is selected from hydrogen and deuterium.

20. The compound of claim 19, wherein R² is H and the compound is selected from any one of the compounds set forth in the table below:

| Cmpd | Z¹ | Y^{a} | Y^{b} | Y^{c} | Y^{d} | Y^{e} | Z² |
|---|---|---|---|---|---|---|---|
| 100 | —CH₃ | D | D | H | H | H | H |
| 101 | —CH₃ | H | H | D | D | D | H |
| 102 | —CH₃ | H | H | H | H | H | D |
| 103 | —CH₃ | D | D | H | H | H | D |
| 104 | —CH₃ | H | H | D | D | D | D |
| 105 | —CH₃ | D | D | D | D | D | H |
| 106 | —CH₃ | D | D | D | D | D | D |
| 107 | —CD₃ | D | D | H | H | H | H |
| 108 | —CD₃ | H | H | D | D | D | H |
| 109 | —CD₃ | H | H | H | H | H | D |
| 110 | —CD₃ | D | D | H | H | H | D |
| 111 | —CD₃ | H | H | D | D | D | D |
| 112 | —CD₃ | D | D | D | D | D | H |
| 113 | —CD₃ | D | D | D | D | D | D |

21. The compound of any one of claims 1 to 20, wherein the compound comprises three or more deuterium atoms.

22. The compound of claim 21, wherein the compound comprises four or more deuterium atoms.

23. The compound of any one of claims 1 to 22, wherein any atom not designated as deuterium is present at its naturally abundant isotopic state.

24. A composition comprising a compound of any one of claims 1 to 23; and an acceptable carrier.

25. The composition of claim 24, wherein the composition is formulated for pharmaceutical administration; and the carrier is a pharmaceutically acceptable carrier.

26. The composition of claim 25, wherein the compound is a compound of claim 7.

27. The composition of claim 25 or 26, wherein the composition is formulated for oral administration.

28. The composition of claim 27 wherein the composition is in the form of a pill, capsule or tablet.

29. A method for the treatment of a neurological disorder, sleep disorder, a sleep/wake disorder, anxiety, depression, or attention deficit disorder, comprising administering to a patient in need of such treatment a composition of claim 25.

30. The method of claim 29, wherein the sleep disorder is selected from primary insomnia, circadian rhythm sleep disorder, dyssomnia NOS, parasomnias, nightmare disorder, sleep terror disorder, narcolepsy, jet lag, sleep apnea, anxiety and substance-induced sleep disorder.

31. The method of claim 30, wherein the sleep disorder is primary insomnia.

32. A method for inducing sleep in a patient in need thereof, comprising administering to the patient a composition of claim 25.

33. A method for inducing sedation, hypnosis or skeletal muscle relaxation in a patient in need thereof, comprising administering to the patient a composition of claim 25.

34. The method of claim 29, wherein the sleep disorder is substance induced insomnia.

35. The method of claim 34 wherein the substance is selected from caffeine, alcohol, amphetamine, an opioid, a sedative, a hypnotic, and an anxiolytic.

36. The method of claim 29 wherein the compound is administered orally.

37. A method of modulating a GABA receptor-chloride ionophore complex in a cell through binding to the neurosteroid site on the complex, comprising contacting the cell with a compound of claim 1.

38. A pharmaceutical composition for use in the treatment of a neurological disorder, sleep disorder, a sleep/wake disorder, anxiety, depression, or attention deficit disorder, comprising a compound of any one of claims 1 to 23; and a pharmaceutically acceptable carrier.

39. The pharmaceutical composition of claim 38, wherein said composition is used in the treatment of a sleep disorder.

40. The pharmaceutical composition of claim 39, wherein said composition is used in the treatment of insomnia.
